(19) United States
(12) Patent Application Publication
(10) Pub. No.: US 2026/0134976 A1
(43) Pub. Date: May 14, 2026

(54) SYSTEMS AND METHODS FOR MAGNETIC RESONANCE BASED RECONSTRUCTION OF ELECTRICAL PROPERTIES

(71) Applicants: NEW YORK UNIVERSITY, New York, NY (US); THE REGENTS OF THE UNIVERSITY OF CALIFORNIA, Oakland, CA (US); MASSACHUSETTS INSTITUTE OF TECHNOLOGY, Cambridge, MA (US)

(72) Inventors: Xinling YU, Santa Barbara, CA (US); Ziyue LIU, Santa Barbara, CA (US); Zheng ZHANG, Santa Barbara, CA (US); José E.C. SERRALLES, Cambridge, MA (US); Ilias I. GIANNAKOPOULOS, New York, NY (US); Luca DANIEL, Cambridge, MA (US); Riccardo LATTANZI, New York, NY (US)

(73) Assignees: NEW YORK UNIVERSITY, New York, NY (US); THE REGENTS OF THE UNIVERSITY OF CALIFORNIA, Oakland, CA (US); MASSACHUSETTS INSTITUTE OF TECHNOLOGY, Cambridge, MA (US)

(21) Appl. No.: 19/120,698

(22) PCT Filed: Oct. 16, 2023
(86) PCT No.: PCT/US2023/077017

§ 371 Date: Apr. 11, 2025

Related U.S. Application Data
(60) Provisional application No. 63/379,628, filed on Oct. 14, 2022.

(51) Int. Cl. G16H 30/20; G16H 30/40; G16H 50/20; G16H 50/70

(52) U.S. Cl. CPC G16H 30/20; G16H 30/40; G16H 50/20; G16H 50/70

(57) ABSTRACT

A method can include providing a first neural network connected to a second neural network. The first neural network can represent B⁺₁ and the second neural network can represent electrical properties. The method can include training the first neural network and the second neural network jointly. The method can include determining, from the trained first neural network and the trained second neural network B⁺₁, a prediction of and electrical properties at one or more predetermined locations. The method can include outputting the prediction of B⁺₁ and EP.

### CROSS-REFERENCE TO RELATED PATENT APPLICATIONS

This application claims the benefit and priority of U.S. Provisional Patent Application No. 63/379,628, filed on Oct. 14, 2022, the entirety of which is incorporated by reference herein.

### GOVERNMENT RIGHTS

This invention was made with Governmental support under Contract No. R01 EB024536 awarded by the National Institute of Health (NIH) and NSF2107321 awarded by the National Science Foundation (NSF). The government has certain rights in the invention.

### TECHNICAL FIELD

The present disclosure relates generally to electrical property maps, more particularly, the present disclosure relates to systems and methods using machine learning to reconstruct electrical property maps based on magnetic resonance based electrical properties tomography (MR-EPT) and for estimating electrical properties from noisy or incomplete magnetic resonance measurements.

### BACKGROUND

Electrical properties (EP) can include permittivity and electric conductivity.

### SUMMARY

At least one aspect of the present disclosure is directed to a method. The method can include providing a first neural network connected to a second neural network. The first neural network can represent

$${B}_{1}^{+}$$

and the second neural network can represent electrical properties. The method can include training the first neural network and the second neural network jointly. The method can include determining, from the trained first neural network and the trained second neural network, a prediction of

$${B}_{1}^{+}$$

and electrical properties at one or more predetermined locations. The method can include outputting the prediction

$${B}_{1}^{+}$$

### and EP.

Another aspect of the present disclosure is directed to a non-transitory computer-readable media having computer-readable instructions stored thereon that when executed by a processor cause the processor to provide a first neural network connected to a second neural network, the first neural network representing

$${B}_{1}^{+}$$

and the second neural network representing electrical properties (EP), train the first neural network and the second neural network jointly, determine, from the trained first neural network and the trained second neural network, a prediction of

$${B}_{1}^{+}$$

and EP at one or more predetermined locations, and output the prediction of

$${B}_{1}^{+}$$

### and EP.

Another aspect of the present disclosure is directed to a method for identifying a medical indication. The method can include obtaining magnetic resonance (MR) data for an individual. The method can include constructing an electrical property (EP) map for the individual using a machine learning model. The method can include identifying a medical condition in the individual based on a correlation between the EP map and medical indications.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates a method of utilizing machine learning models to de-noise and/or construct EP maps for an individual, according to an example implementation.

FIG. 2 illustrates an exemplary computing device that can perform processes, according to an example implementation.

FIG. 3 illustrates a schematic of a distributed computing devices, according to an example implementation.

FIGS. 4A-4E illustrate exemplary data, according to an example implementation.

FIG. 5 illustrates a network architecture of

$${B}_{1}^{+} ,$$

according to an example implementation.

FIG. 6 illustrates a network architecture of electrical properties, according to an example implementation.

FIG. 7 illustrates reconstruction from noisy measurements, according to an example implementation.

FIG. 8 illustrates reconstruction from incomplete measurements, according to an example implementation.

FIG. 9 illustrates a birdcage coil at 3T, according to an example implementation.

FIG. 10 illustrates reconstruction, according to an example implementation.

FIG. 11 illustrates physics-informed Fourier networks for electrical properties tomography (PIFON-EPT), according to an example implementation.

FIG. 12 illustrates EP reconstruction with simplified PIFON-EPT for a uniform dielectric cylinder, according to an example implementation.

FIG. 13 illustrates EP reconstruction with generalized PIFON-EPT for a uniform dielectric cylinder, according to an example implementation.

FIG. 14 illustrates reconstructed

$${B}_{1}^{+}$$

with simplified PIFON-EPT inside a uniform dielectric cylinder, according to an example implementation.

FIG. 15 illustrates reconstructed

$${B}_{1}^{+}$$

with generalized PIFON-EPT inside a uniform dielectric cylinder, according to an example implementation.

FIG. 16 illustrates the geometry of the high-pass birdcage coil loaded with a two-compartments cylindrical phantom, according to an example implementation.

FIG. 17 illustrates EP reconstructed with generalized PIFON-EPT for the two-compartment cylindrical phantom, according to an example implementation.

FIG. 18 illustrates reconstructed

$${B}_{1}^{+}$$

with generalized PIFON-EPT inside a uniform compartment cylindrical phantom, according to an example implementation.

FIG. 19 illustrates conductivity reconstructed with phase-based H-EPT for the two-compartment cylindrical phantom, according to an example implementation.

FIG. 20 illustrates conductivity reconstructed with phase-based CR-EPT for the two-compartment cylindrical phantom, according to an example implementation.

FIG. 21 illustrates noisy synthetic

$${\tilde{B}}_{1}^{+}$$

measurements, according to an example implementation.

FIG. 22 illustrates EP reconstructed with simplified PIFON-EPT for the four-compartment phantom, according to an example implementation.

FIG. 23 illustrates reconstructed

$${B}_{1}^{+}$$

with simplified PIFON-EPT for the four-compartment phantom, according to an example implementation.

FIG. 24 illustrates incomplete noisy synthetic

$${\tilde{B}}_{1}^{+}$$

measurements with 50% of the voxels set to zero, according to an example implementation.

FIG. 25 illustrates reconstructed EP with simplified PIFON-EPT for the incomplete four-compartment phantom, according to an example implementation.

FIG. 26 illustrates reconstructed

$${B}_{1}^{+}$$

with simplified PIFON-EPT for the incomplete four-compartment phantom, according to an example implementation.

FIG. 27 illustrates a method for electrical property tomography, according to an example implementation.

Like reference numbers and designations in the various drawings indicate like elements.

### DETAILED DESCRIPTION

Following below are more detailed descriptions of various concepts related to, and implementations of, methods, apparatuses, and systems for estimating electrical properties from noisy or incomplete magnetic resonance measurements. The various concepts introduced above and discussed in greater detail below may be implemented in any of a number of ways, as the described concepts are not limited to any particular manner of implementation. Examples of specific implementations and applications are provided primarily for illustrative purposes.

The systems and methods of the present disclosure can include a physics-informed deep learning framework for EP reconstruction that is able to remove the amplification of the noise that appears in differential methods. Electrical properties (EP) and clean

$${B}_{1}^{+}$$

can be reconstructed from incomplete noisy simulated measurements. The network can work for high levels of noise (e.g., peak SNR=20). The method can be easily combined with other MR-based reconstruction methods such as convection-reaction EPT.

The systems and methods of the present disclosure can also include Physics-Informed Fourier Networks for Electrical Properties (EP) Tomography (PIFON-EPT). PIFON-EPT can include a deep learning-based method for EP reconstruction using noisy and/or incomplete magnetic resonance (MR) measurements. This approach can leverage the Helmholtz equation to constrain two networks, responsible for the denoising and completion of the transmit fields, and the estimation of the object's EP, respectively. A random Fourier features mapping can be embedded into the networks to enable efficient learning of high-frequency details encoded in the transmit fields. The efficacy of PIFON-EPT can be demonstrated through several simulated experiments at 3 and 7 tesla MR imaging. The method can reconstruct physically consistent EP and transmit fields. Specifically, when only 20% of the noisy measured fields were used as inputs, PIFON-EPT reconstructed the EP of a phantom with ≤5% error, and denoised and completed the measurements with ≤1% accuracy. Additionally, PIFON-EPT can be adapted to solve the generalized Helmholtz equation that accounts for gradients of EP between inhomogeneities. This yielded improved results at interfaces between different materials without explicit knowledge of boundary conditions. PIFON-EPT can simultaneously reconstruct EP and transmit fields from incomplete noisy MR measurements.

Electrical properties (EP), namely permittivity and electric conductivity, can dictate the interactions between electromagnetic waves and biological tissue. EP can be potential biomarkers for pathology characterization, such as cancer, and improve therapeutic modalities, such radiofrequency hyperthermia and ablation. Magnetic resonance (MR)-based electrical properties tomography (MR-EPT) can use MR measurements to reconstruct the EP maps. Using the homogeneous Helmholtz equation, EP can be directly computed through calculations of second order spatial derivatives of the measured magnetic transmit or receive fields

$$( {B}_{1}^{+} , {B}_{1}^{-} ) .$$

However, the numerical approximation of derivatives can lead to noise amplifications in the measurements and thus erroneous reconstructions. A noise-robust supervised learning-based method (DL-EPT) was introduced for EP reconstruction. (See e.g., Mandija, Stefano, et al. “Opening a new window on MR-based electrical properties tomography with deep learning.” Scientific reports 9.1 (2019): 1-9; the disclosure of which is hereby incorporated by reference in its entirety.) However, the pattern-matching nature of such network does not allow it to generalize for new samples since the network's training is done on a limited number of simulated data. Thus, there is a need in the art for a robust method for EP map construction using MR data.

Embodiments herein leverage recent developments on physics-informed deep learning to solve the Helmholtz equation for the EP reconstruction. (See e.g., Raissi, Maziar, et al. “Physics-informed neural networks: A deep learning framework for solving forward and inverse problems involving nonlinear partial differential equations.” Journal of Computational physics 378 (2019): 686-707; the disclosure of which is hereby incorporated by reference in its entirety.) Many embodiments use deep neural network (NN) algorithms constrained by the Helmholtz equation to effectively de-noise

$${B}_{1}^{+}$$

measurements and reconstruct EP directly at an arbitrarily high spatial resolution without requiring any known

$${B}_{1}^{+}$$

and EP distribution pairs.

When assuming a smooth distribution of the EP, the relationship between the transmit fields and the underlying EP can be described by the Helmholtz equation:

$${∇}^{2} {B}_{1}^{+} + {k}_{0}^{2} {ε}_{c} {B}_{1}^{+} = 0 ,$$

where k₀ is the wave number in free space and ε_{c} is the relative complex permittivity. Various embodiments de-noise the measured complex transmit field

$${\{ ( {r}_{i} , {\tilde{B}}_{1}^{+} ( {r}_{i} ) ) \}}_{i = 1}^{N}$$

at N 3D locations rᵢ to reconstruct the EP. Many embodiments accomplish this task by employing prior physical knowledge as a constraint in processing the noisy measurements. Such knowledge can include defining a NN

$${B}_{1}^{+} ( r ; {θ}_{1} ) ,$$

parameterized by a set of weights and biases θ₁, to estimate the complex

$${B}_{1}^{+}$$

fields. Additional embodiments use NN ε_{c}(r;θ₂) with independent weights and biases θ₂ to estimate a distribution of relative complex permittivity. Combining both NN, the Helmholtz residual then takes the form:

$${ℛ}_{H} = {∇}^{2} {ℬ}_{1}^{+} ( r ; {θ}_{1} ) + {k}_{0}^{2} {ε}_{c} ( r ; {θ}_{2} ) {ℬ}_{1}^{+} ( r ; {θ}_{1} ) \qquad Eq . 1$$

In Equation 1, the Laplacian of the NN representation can be readily computed to machine precision using automatic differentiation. (See e.g., Baydin, Atilim Gunes, et al. “Automatic differentiation in machine learning: a survey.” Journal of Machine Learning Research 18 (2018): 1-43; the disclosure of which is hereby incorporated by reference in its entirety.) Many embodiments obtain candidate parameters {θ₁, θ₂} via gradient descent using a composite loss function that aims to fit the measured

$${B}_{1}^{+}$$

field while also penalizing the Helmholtz equation residual:

$$ℒ ( {θ}_{1} , {θ}_{2} ) = {ℒ}_{data} ( {θ}_{1} ) + {λℒ}_{r} ( {θ}_{1} , {θ}_{2} ) , \\ where {ℒ}_{r} ( {θ}_{1} , {θ}_{2} ) = \frac{1}{N} {\sum }_{i = 1}^{N} {\vert {ℛ}_{H} ( {r}_{i} , {θ}_{1} , {θ}_{2} ) \vert}^{2} , and \qquad Eq . 2$$

$${ℒ}_{data} ( {θ}_{1} ) = \frac{1}{N} {\sum }_{i = 1}^{N} {\vert Re \{ {ℬ}_{1}^{+} ( {r}_{i} ; {θ}_{1} ) \} - Re \{ {\tilde{ℬ}}_{1}^{+} ( {r}_{i} ) \} \vert}^{2} + {\vert Im \{ {ℬ}_{1}^{+} ( {x}_{i} ; {θ}_{1} ) \} - Im \{ {\tilde{ℬ}}_{1}^{+} ( {x}_{i} ) \} \vert}^{2} .$$

In many embodiments,

$${B}_{1}^{+}$$

and ε_{c} are independent NNs. In certain embodiments, the independent NNs are connected, including partial and fully connected. The NNs can have any number of suitable layers and/or units per hidden layer, including 2, 3, 5, 10, 15, or more layers and 16, 64, 128, 256, 512, or more. Additionally, activation functions can vary depending on NN, with some embodiments using a sine activation function. Various embodiments train the NNs jointly, such as by minimizing loss of equation 2. Certain embodiments use the Adam optimizer for any number of iterations for robust use of the model, such as 60 k iterations, 90 k iterations, 120 k iterations or more. Learning rates can be set to any suitable rate, such as 10⁻², 10⁻³, 10⁻⁴, 10⁻⁵, 10⁻⁶, or lower. Some embodiments set a decaying schedule of learning rates, such that the learning rate decreases after a set number of iterations—for example, some embodiments comprise a decaying schedule of learning rates 10⁻³, 10⁻⁴, 10⁻⁵, decreased after every 40 k iterations (i.e., 120 k iterations total).

### Methods of Use

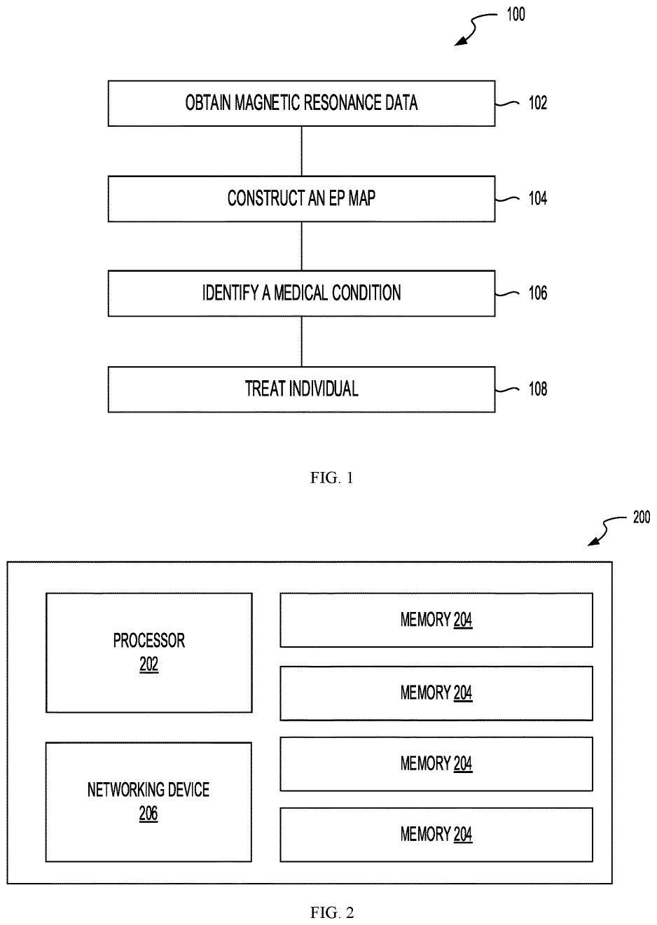

As noted previously, reconstructing EP maps can provide insights as biomarkers for pathology characterization, such as cancer, and improve therapeutic modalities, such radiofrequency hyperthermia and ablation. Thus, some embodiments utilize machine learning methodologies, such as those described herein as a diagnostic, prognostic, and/or other tool for guiding medical care. FIG. 1 provides a method 100 of utilizing machine learning models to de-noise and/or construct EP maps for an individual.

At 102, many embodiments obtain magnetic resonance (MR) data from an individual. In various embodiments, the MR data is obtained from MR imaging (MRI). In further embodiments MR data is obtained from previously imaged and gathered data, such as stored on a server, hard drive, and/or other storage media. Such access can be local and/or remote (e.g., obtained via a network).

Various embodiments, at 104, construct an EP map. Embodiments can construct the EP map via methods, process, and/or systems such as those described herein.

Further embodiments identify a disease and/or disorder based on the EP map at 106. Many such embodiments utilize one or more biomarkers identified in the EP map as an indicator of such a disease and/or disorder. Some embodiments utilize a machine learning model, method, and/or system trained to identify diseases, disorders, and/or other medically relevant information based on EP maps.

At 108, many embodiments treat an individual based on the medically relevant information identified in the EP maps. Such treatment can be rehabilitative, prehabilitative, mitigative, interventive, and/or any other treatment style or technique applicable to the disease, disorder, and/or indication. Treatments can include supplementation, medication, surgery, and/or physical therapy.

The techniques described herein can relate to a physics-informed machine learning model for construction electrical property (EP) maps. The model can include a first neural network to estimate fields from magnetic resonance (MR) data. The model can include a second neural network to estimate to estimate a distribution of relative complex permittivity.

The first neural network and the second neural network can be fully connected. The first neural network and the second neural network can each include 3 hidden layers. Each hidden layer can include 128 units.

The first neural network and the second neural network can be trained by minimizing loss in the equation:

$$ℒ ( {θ}_{1} , {θ}_{2} ) = {ℒ}_{data} ( {θ}_{1} ) + {λℒ}_{r} ( {θ}_{1} , {θ}_{2} ) , \\ where {ℒ}_{r} ( {θ}_{1} , {θ}_{2} ) = \frac{1}{N} {\sum }_{i = 1}^{N} {\vert {ℛ}_{H} ( {r}_{i} , {θ}_{1} , {θ}_{2} ) \vert}^{2} , and$$

$${ℒ}_{data} ( {θ}_{1} ) = \frac{1}{N} {\sum }_{i = 1}^{N} {\vert Re \{ {ℬ}_{1}^{+} ( {r}_{i} ; {θ}_{1} ) \} - Re \{ {\tilde{ℬ}}_{1}^{+} ( {r}_{i} ) \} \vert}^{2} + {\vert Im \{ {ℬ}_{1}^{+} ( {x}_{i} ; {θ}_{1} ) \} - Im \{ {\tilde{ℬ}}_{1}^{+} ( {x}_{i} ) \} \vert}^{2} .$$

Training can utilize an optimizer. For example, the training can utilize the Adam optimizer. The first neural network and the second neural network can be trained for 120,000 iterations. Training can include a decaying schedule of learning rates 10⁻³, 10⁻⁴, 10⁻⁵, decreased after every 40,000 iterations.

In some aspects, the techniques described herein relate to a method for identifying a medical indication. The method can include obtaining magnetic resonance (MR) data for an individual, constructing an electrical property (EP) map for the individual using a machine learning model, and identifying a medical condition in the individual based on a correlation between the EP map and medical indications.

The machine learning model can include features as described above. The machine learning model can include a first neural network connected to a second neural network. The first neural network can represent

$${B}_{1}^{+}$$

method can include treating the individual based on the identified medical condition.

### Computer Executed Embodiments

Processes that provide the methods and systems for determining image quality in accordance with some embodiments are executed by a computing device or computing system, such as a desktop computer, tablet, mobile device, laptop computer, notebook computer, server system, and/or any other device capable of performing one or more features, functions, methods, and/or steps as described herein. The relevant components in a computing device that can perform the processes in accordance with some embodiments are shown in FIG. 2. One skilled in the art will recognize that computing devices or systems may include other components that are omitted for brevity without departing from described embodiments. A computing device 200 in accordance with such embodiments comprises a processor 202 and at least one memory 204. Memory 204 can be a non-volatile memory and/or a volatile memory, and the processor 202 is a processor, microprocessor, controller, or a combination of processors, microprocessor, and/or controllers that performs instructions stored in memory 204. Such instructions stored in the memory 204, when executed by the processor, can direct the processor, to perform one or more features, functions, methods, and/or steps as described herein. Any input information or data can be stored in the memory 204—either the same memory or another memory. In accordance with various other embodiments, the computing device 200 may have hardware and/or firmware that can include the instructions and/or perform these processes.

Certain embodiments can include a networking device 206 to allow communication (wired, wireless, etc.) to another device, such as through a network, near-field communication, Bluetooth, infrared, radio frequency, and/or any other suitable communication system. Such systems can be beneficial for receiving data, information, or input (e.g., images) from another computing device and/or for transmitting data, information, or output (e.g., quality score, rating, etc.) to another device. In various embodiments, the networking device can be used to send and/or receive update models, interfaces, etc. to a user device.

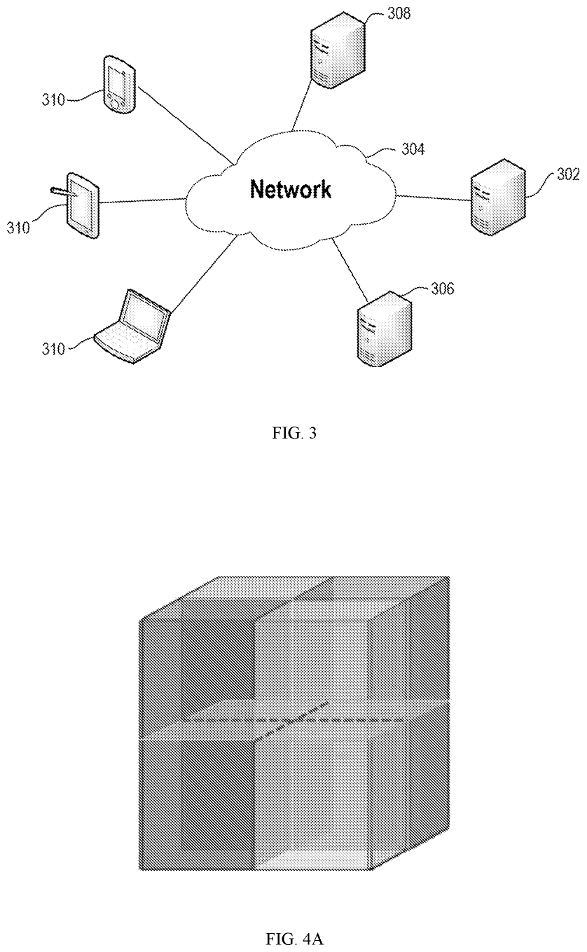

Turning to FIG. 3, an embodiment with distributed computing devices is illustrated. Such embodiments may be useful where computing power is not possible at a local level, and a central computing device (e.g., server) performs one or more features, functions, methods, and/or steps described herein. In such embodiments, a computing device 302 (e.g., server) is connected to a network 304 (wired and/or wireless), where it can receive inputs from one or more computing devices, including data from a records database or repository 306, data provided from a laboratory computing device 308, and/or any other relevant information from one or more other remote devices 310. Once computing device 302 performs one or more features, functions, methods, and/or steps described herein, any outputs can be transmitted to one or more computing devices 306, 308, 310 for entering into records.

In accordance with still other embodiments, the instructions for the processes can be stored in any of a variety of non-transitory computer readable media appropriate to a specific application.

### EXEMPLARY EMBODIMENTS

Although the following embodiments provide details on certain embodiments of the inventions, it should be understood that these are only exemplary in nature, and are not intended to limit the scope of the invention.

### Example 1: Machine Learning Based Reconstruction of EP

Machine learning methodologies have the potential to reconstruct EP maps from magnetic resonance data.

This exemplary embodiment can utilize many components of the methods described herein. Specifically,

$${B}_{1}^{+}$$

and ε_{c} were two independent, fully connected NNs with 3 layers, 128 units per hidden layer, and a sine activation function. The NNs were trained jointly by minimizing loss of equation 2 using the Adam optimizer for 120 k iterations in total, with a decaying schedule of learning rates 10⁻³, 10⁻⁴, 10⁻⁵, decreased after every 40 k iterations.

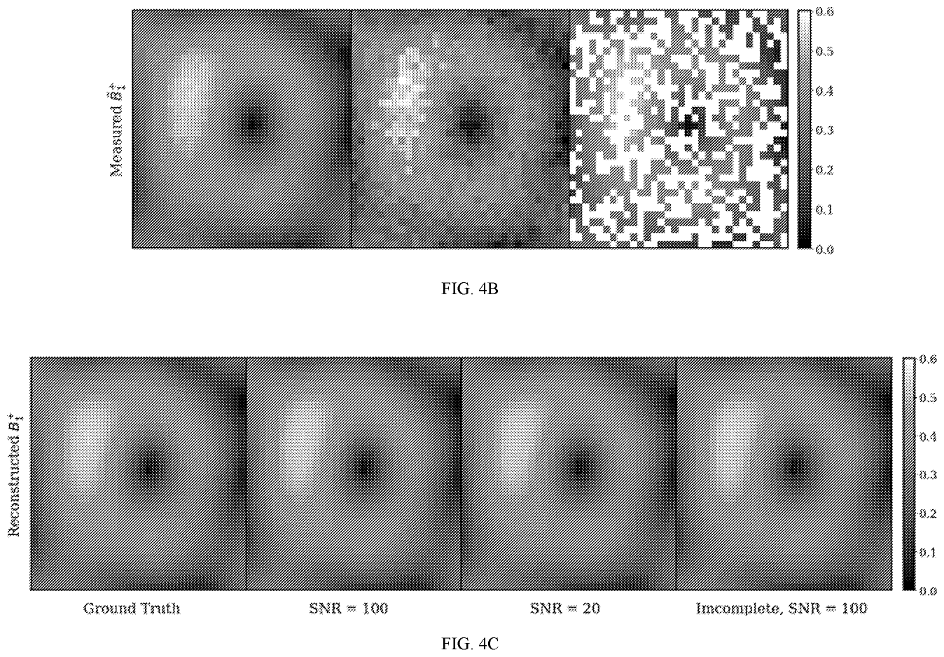

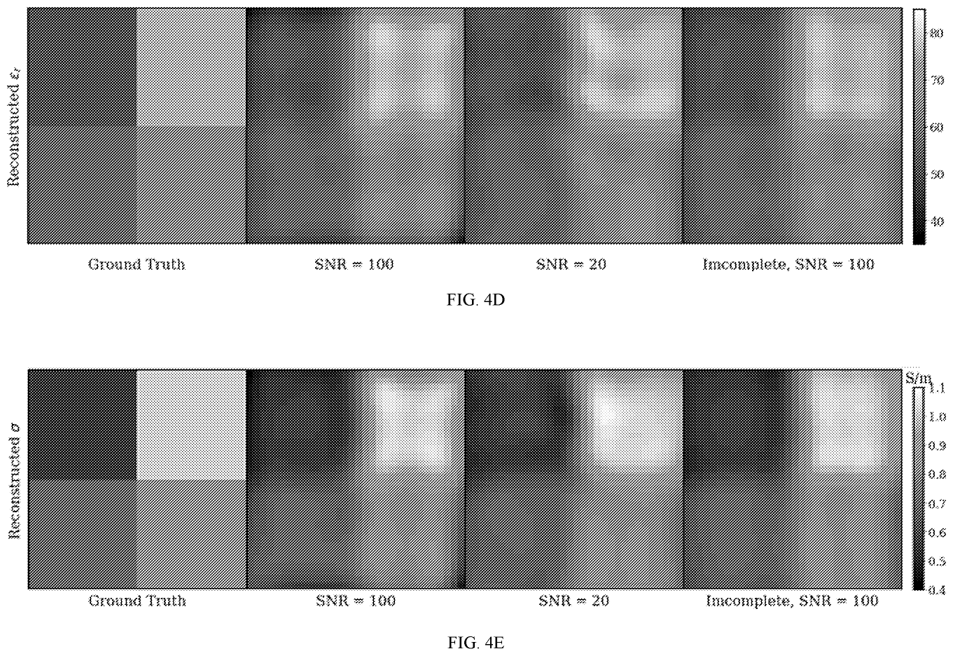

This embodiment considered a 4-compartment phantom, such as illustrated in FIG. 4A with permittivity ∈ᵣ={56, 51, 65, 76} and electric conductivity σ={0.69, 0.56, 0.84, 1.02}. This embodiment used one external excitation to generate synthetic transmit fields map inside the phantom at 7 tesla MRI frequency. For this embodiment, set λ=10⁻⁶ in equation 2 was set to make each component of the loss approximately equal at the beginning of training. Once trained, the resulting physics-informed NN

$${B}_{1}^{+} ( r ; {θ}_{1} )$$

and ε_{c}(r;θ₂) can be used to obtain physically consistent predictions of

$${B}_{1}^{+}$$

and EP at any arbitrary 3D location. Three noisy

$${\tilde{B}}_{1}^{+}$$

maps are shown in FIG. 4B, where (from left to right)

$${\tilde{B}}_{1}^{+}$$

measurements (peak SNR=100, 200), incomplete

$${\tilde{B}}_{1}^{+}$$

measurements (peak SNR=100). FIGS. 4C-4E illustrate denoised

$${B}_{1}^{+}$$

maps and the reconstructed EP for the central axial cut of the phantom. FIG. 4C provides ground truth

$${B}_{1}^{+}$$

compared with (from left to right) denoised

$${B}_{1}^{+}$$

measurements (peak SNK=100, 20), reconstructed

$${B}_{1}^{+}$$

for incomplete

$${B}_{1}^{+}$$

measurements (SNR=100). FIG. 4D provides ground truth εᵣ compared with (from left to right) reconstructed εᵣ (SNR=100, 20), reconstructed εᵣ using incomplete

$${B}_{1}^{+}$$

measurements (SNR=100). FIG. 4E illustrates ground truth σ compared with (from left to right) reconstructed σ (SNR=100, 20), reconstructed σ using incomplete

$${B}_{1}^{+}$$

measurements (SNR=100).

The average value of the peak normalized absolute error (PNAE) over the entire volume is 0.23%, 3.52%, and 4.45% for the

$${B}_{1}^{+} ,$$

relative permittivity and conductivity, respectively, when peak SNR=100; The error is found to be 0.44%, 2.55% and 3.43% for the

$${B}_{1}^{+} ,$$

relative permittivity and conductivity, respectively, when peak SNR=20. These results show that the algorithm is accurate and robust for a significant amount of noise. Additionally, the algorithm can also reconstruct the EP using incomplete measurements. For example, when half of the

$${\tilde{B}}_{1}^{+}$$

measurements of the entire volume are missing, as in FIG. 4B right, this embodiment can still reconstruct the

$${B}_{1}^{+}$$

and the EP in the whole domain of interest, in which case the error is 0.24%, 2.49% and 3.68% for the

$${B}_{1}^{+} ,$$

relative permittivity and conductivity, respectively.

This embodiment provides a physics-informed deep learning framework that is able to de-noise simulated

$${B}_{1}^{+}$$

measurements and provide accurate EP reconstructions of an inhomogeneous phantom. This is the first time that deep learning can reconstruct the

$${B}_{1}^{+}$$

from incomplete noisy measurements, which shows the potential to improve other MR-based reconstruction methods. The usage of multiple

$${B}_{1}^{+}$$

magnitudes and relative phases (measurable in MRI) can be explored and the assumption of knowing the absolute phase can be discarded.

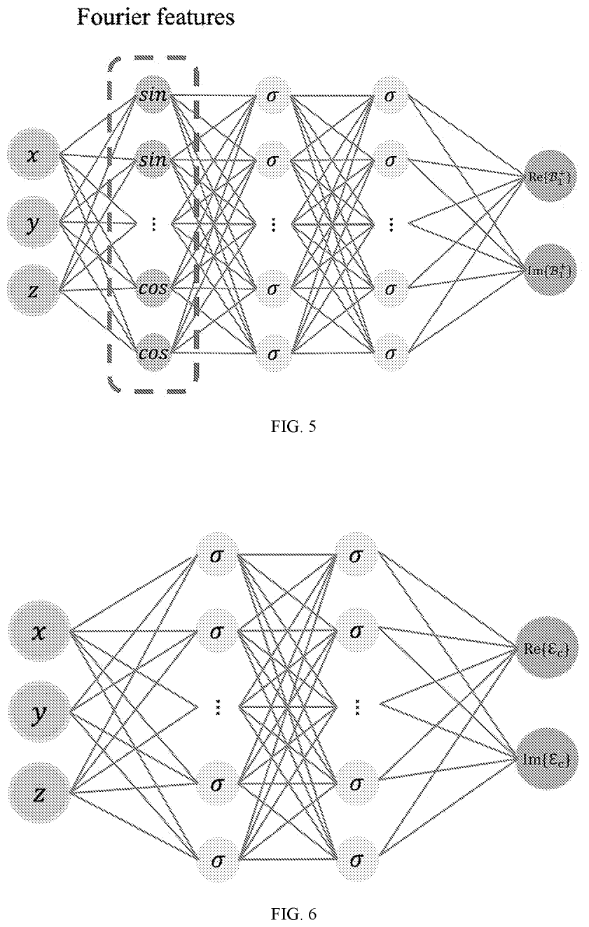

FIG. 5 illustrates a network architecture of

$${B}_{1}^{+} . {B}_{1}^{+} ( r ; {θ}_{1} )$$

can be constructed using a random Fourier feature mapping described by Equation 3:

$$γ ( r ) = [ \begin{matrix}cos ( Br ) \\ sin ( Br )\end{matrix} ] \qquad Eq . 3$$

as a coordinate embedding of the inputs, followed by a fully-connected neural network.

FIG. 6 illustrates a network architecture of electrical properties (e.g., ε_{c}). ε_{c}(r;θ₂) can be a fully-connected neural network that outputs the distribution of EP.

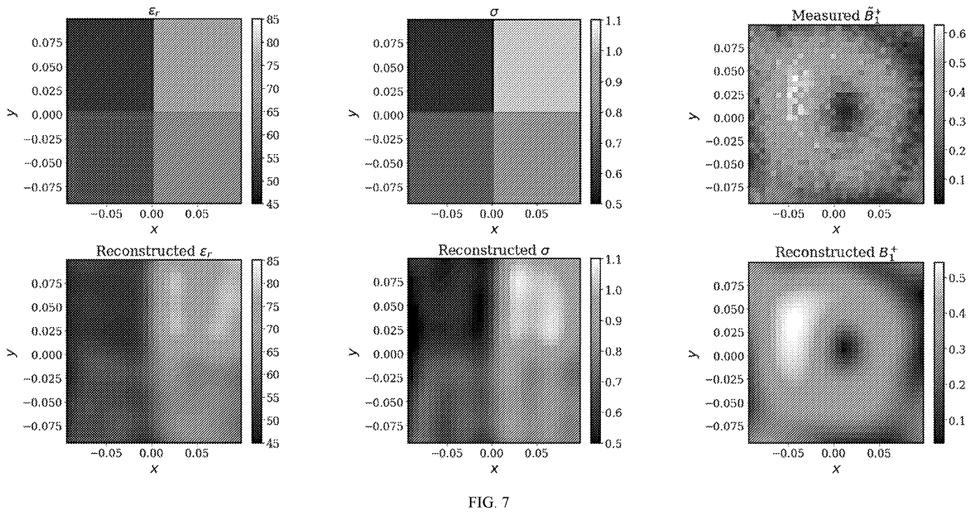

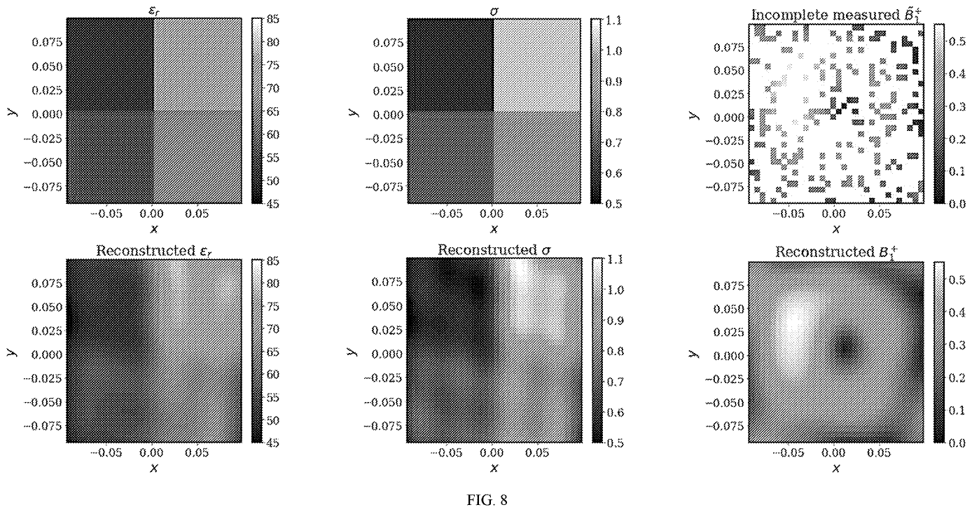

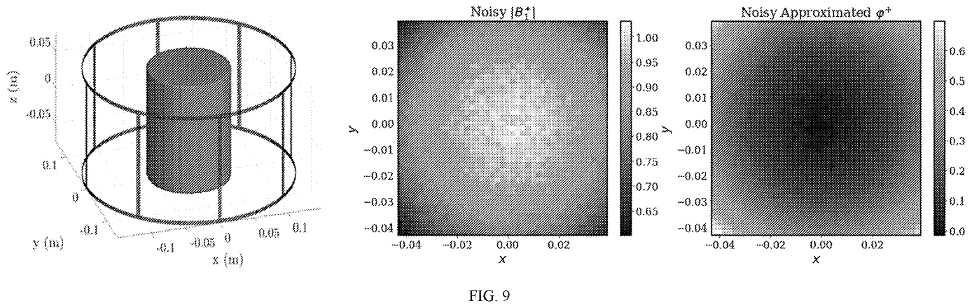

FIG. 7 illustrates reconstruction from noisy measurements. The average error can be 2.57% (Er), 3.96% (g), and 0.49%

$$( {B}_{1}^{+} ) .$$

The peak error can be 16.25% (εᵣ), 33.45% (σ) and 4.02% (B⁺₁). For the measured

$${\tilde{B}}_{1}^{+} ,$$

80 the peak SNR can be 20.

FIG. 8 illustrates reconstruction from incomplete measurements. The average error can be 2.77% (εᵣ), 4.06% (σ) and 0.57%

$$( {B}_{1}^{+} ) .$$

The peak error can be 18.06% (εᵣ), 34.12% (σ) and 6.11%

$$( {B}_{1}^{+} ) .$$

For the incomplete measured

$${\tilde{B}}_{1}^{+} ,$$

80% of the measurements can be missing and the peak SNR can be 50.

FIG. 9 illustrates a birdcage coil at 3T. The data can include noisy

$$\vert {B}_{1}^{+} \vert$$

and transceive phase φ=φ⁺+φ⁻ (all measurable in MRI). The complex

$${B}_{1}^{+}$$

can be approximated by

$$\vert {B}_{1}^{+} \vert exp ( \frac{i φ}{2} )$$

and then used for reconstruction. The birdcage coil can have a 2 mm resolution. The cylinder can have a radius of 6 cm and a length of 14 cm. The homogenous EP distribution can be εᵣ=80 and σ=0.8. The 8 cm×8 cm×2 cm (18,491 voxels) measurement at the center of the cylinder can be extracted. The peak SNR can be 50.

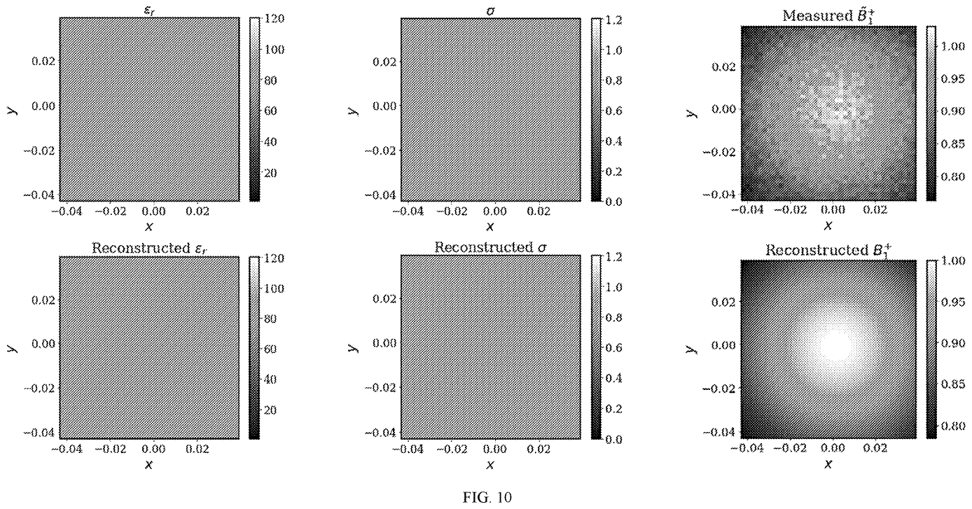

FIG. 10 illustrates reconstruction. The average error can be 1.41% (εᵣ), 0.99% (σ) and 0.10%

$$( {B}_{1}^{+} ) .$$

The peak error can be 2.04% (εᵣ), 1.70% (σ) and 0.57%

$$( {B}_{1}^{+} ) .$$

### Example 2: MR-Based Electrical Property Tomography

Electrical properties (e.g., relative permittivity and electric conductivity) can determine the interactions between electromagnetic waves and biological tissue. Electrical properties can be employed as biomarkers for pathologies such as cerebral ischemia and cancer. Electrical properties can also be used to improve the effectiveness of therapeutic modalities such as radiofrequency hyperthermia.

EP tomography (EPT) methods can be based on MR measurements, such as the magnetic transmit

$$( {B}_{1}^{+} )$$

or receive

$$( {B}_{1}^{-} )$$

field maps. techniques can be classified based on the form of Maxwell's equations (e.g., differential or integral) they use to fit the MR measurements. Differential methods, such as the Helmholtz EPT (H-EPT) or the convection-reaction EPT (CR-EPT) can require the calculations of spatial derivatives of noisy measured

$$( {B}_{1}^{+} )$$

maps, which can lead to errors and artifacts in the reconstructions. On the other hand, integral equation-based methods can be robust to noise, but can require computationally expensive iterative optimizations that rely on an accurate model of the transmit coils and fine-tuned regularization parameters.

Data-driven deep learning-based methods can be used for EP reconstruction. These methods can treat MR measurements and EP distributions as 2D images or 3D volumes, and train regression convolution neural networks as surrogate EP reconstruction models from simulated training data. These supervised learning-based techniques can perform well in simulation, but they may not reliable in vivo due to the limited number of different cases included in the training data. To improve the generalization to in vivo data, hybrid techniques can embed deep learning into EP mapping methods. These hybrid methods can use neural networks to generate initial guesses of EP for iterative reconstruction schemes, or diffusion and convection coefficients for the convection-reaction equation. While these approaches can improve generalization, several electromagnetic simulations may still be required to generate training data, which can be very expensive and time-consuming. A hybrid technique can directly reconstruct conductivity from input transceive phases. In such a method, a neural network can be trained to represent the input transceive phase map, where the gradients of the phase are computed by automatic differentiation and then used to solve the phase-only convection-reaction EPT. The reconstructed conductivity can be compared with ground-truth values at the boundary, as a regularization for the neural network that represents the phase. Since this method can retain the physics of EPT, it may not require a comprehensive set of electromagnetic simulations. However, learning a single neural network that can simultaneously represent the ground-truth phase and provide accurate gradient approximations directly from noisy measured phase maps can be challenging, because they can yield highly inaccurate EP reconstructions in most cases.

The systems and methods of the present disclosure can include the Physics-Informed Fourier Networks (PIFONs) Electrical Properties Tomography (PIFON-EPT) framework. PIFON-EPT can leverage developments on physics-informed deep learning, and Fourier features mapping to learn both the EP distribution and the

$$( {B}_{1}^{+} )$$

field globally from noisy and/or incomplete

$${\tilde{B}}_{1}^{+}$$

measurements. The proposed framework can efficiently de-noise the

$$( {B}_{1}^{+} )$$

measurements and provide physically consistent reconstructions of the EP. Differently from other supervised learning-based EPT methods, the PIFON-EPT technique can reconstruct EP directly, without being trained on known

$$( {B}_{1}^{+} )$$

and EP distribution pairs. Compared with physics-aware hybrid EPT methods in which EP are still solved numerically from convection-reaction equation with boundary condition, the present method can represent EP as a neural network constrained by the Helmholtz equations and does not require any prior EP information.

The relation between the magnetic field (B) and the EP of a medium can be described by the Helmholtz equation:

$${∇}^{2} B + {k}_{0}^{2} {ε}_{c} B + ∇ {ε}_{c} \times \frac{∇ \times B}{{ε}_{c}} = 0 , \qquad Eq . 4$$

where k₀ is the wave number in vacuum and

$${ε}_{c} = {ε}_{r} - \frac{σ}{{ωε}_{0}} , \qquad Eq . 5$$

is the relative complex permittivity. Here, εᵣ is the relative permittivity and σ is the electric conductivity, [symbol] denotes the imaginary unit, ω denotes the angular frequency, and ε₀ denotes the vacuum permittivity. Since the full transmit B₁ cannot be measured in an MRI scanner, but only its positively rotating component

$${B}_{1}^{+} = ( {B}_{x} + {B}_{y} ) / 2 ,$$

Eq. 4 can be rewritten with the help of Gauss' law (∇·B=0) as:

$${∇}^{2} {B}_{1}^{+} + {k}_{0}^{2} {ε}_{c} {B}_{1}^{+} = ( \frac{\partial {B}_{1}^{+}}{\partial x} - \frac{\partial {B}_{1}^{+}}{\partial y} + \frac{1}{2} \frac{\partial {B}_{Z}}{\partial z} ) ( {g}_{x} + {g}_{y} ) + ( \frac{\partial {B}_{1}^{+}}{\partial z} - \frac{1}{2} \frac{\partial {B}_{z}}{\partial x} + \frac{1}{2} \frac{\partial {B}_{Z}}{\partial y} ) {g}_{z} \qquad Eq . 6$$

Here, g:=(gₓ,g_{y},g_{z}):=∇ ln ε_{c}. If a smooth distribution of the EP is assumed, their gradient g can be ignored, and Eq. 6 becomes the homogeneous Helmholtz equation:

$${∇}^{2} {B}_{1}^{+} + {k}_{0}^{2} {ε}_{c} {B}_{1}^{+} = 0 \qquad Eq . 7$$

Equations 6 and 7 can be solved for the EP, starting from measured

$${B}_{1}^{+}$$

maps. Methods based on this approach can include the Helmholtz EPT and the convection-reaction EPT.

For the Helmholtz EPT, a homogeneous distribution of the EP and access to measure complex

$${\tilde{B}}_{1}^{+}$$

maps can be assumed. The homogeneous Helmholtz equation (Eq. 7) can be inverted to estimate the EP:

$${ε}_{c} = - \frac{{∇}^{2} {\tilde{B}}_{1}^{+}}{{k}_{0}^{2} {\tilde{B}}_{1}^{+}} \qquad Eq . 8$$

The second-order spatial derivatives of the measured

$${\tilde{B}}_{1}^{+}$$

can be computed via finite difference approaches. If the measured fields are noisy, smoothing filters such as the 2^{nd} order Savitzky-Golay filter can be applied to improve the numerical derivatives.

For the convection-reaction EPT, high-field MRI scanners (e.g., <7 tesla) can utilize birdcage-based body coils for transmission. In these cases, the B_{z} component of the magnetic field can be assumed negligible near the mid-plane of the magnet bore. As a result, the generalized Helmholtz equation (Eq. 6) can be simplified as:

$${∇}^{2} {\tilde{B}}_{1}^{+} + {k}_{0}^{2} {ℰ}_{c} {\tilde{B}}_{1}^{+} = ( \frac{\partial {\tilde{B}}_{1}^{+}}{\partial x} - \frac{\partial {\tilde{B}}_{1}^{+}}{\partial y} ) ( {g}_{x} + {g}_{y} ) + \frac{\partial {\tilde{B}}_{1}^{+}}{\partial 𝓏} \cdot {g}_{z} \qquad Eq . 9$$

If γ=1/ε_{c}, Eq. 9 can be rewritten as the convection-reaction equation with a zero diffusion term with respect to γ:

$${∇}^{2} {\tilde{B}}_{1}^{+} \cdot γ + {k}_{0}^{2} {\tilde{B}}_{1}^{+} = - ( \frac{\partial {\tilde{B}}_{1}^{+}}{\partial x} - \frac{\partial {\tilde{B}}_{1}^{+}}{\partial y} ) ( \frac{\partial γ}{\partial x} + \frac{\partial γ}{\partial y} ) - \frac{\partial {\tilde{B}}_{1}^{+}}{\partial 𝓏} \cdot \frac{\partial γ}{\partial 𝓏} \qquad Eq . 10$$

By imposing appropriate boundary conditions (for example, the value of γ at the boundary of the domain), the convection-reaction equation (Eq. 10) can be solved with a mesh-based finite difference scheme for γ. As for Helmholtz EPT, also in this case the gradients of the measure

$${\tilde{B}}_{1}^{+}$$

can be estimated using the Savitzky-Golay filter. Since at MRI frequencies below 3 tesla, the absolute phase of

$${B}_{1}^{+}$$

is almost independent nom the permittivity, it is possible to perform conductivity-only reconstructions using only the absolute phase of

$${\tilde{B}}_{1}^{+} ,$$

which, for birdcage coils, can be estimated with the transceive assumption. It is also possible to include an artificial diffusion term to the convection-reaction equation to stabilize and improve the reconstruction results.

The PIFON-EPT can include a deep learning-based frame-work for robust EP estimation using noisy and/or incomplete complex-valued MR measurements. Since in MRI there is no direct access to the absolute phase of

$${B}_{1}^{+} ,$$

one may rely on symmetry assumptions to estimate the complex-valued field in actual experiments. Specifically, at 1.5 and 3 tesla, when RF birdcage coils are used for transmission and reception in quadrature, the

$${B}_{1}^{+} and {B}_{1}^{-}$$

phases are approximately equal. Therefore, since the transceive phase is measurable, the absolute phase of

$${B}_{1}^{+}$$

can be approximated as half the transceive phase. One of the goals of PIFON-EPT can be to learn the EP distributions globally that best describe the complex-valued

$${B}_{1}^{+}$$

at any spatial location (x, y, z) given

$$\{ ( {r}_{i} , {\tilde{B}}_{1}^{+} ( {r}_{i} ) ) \} \frac{N}{i = 1}$$

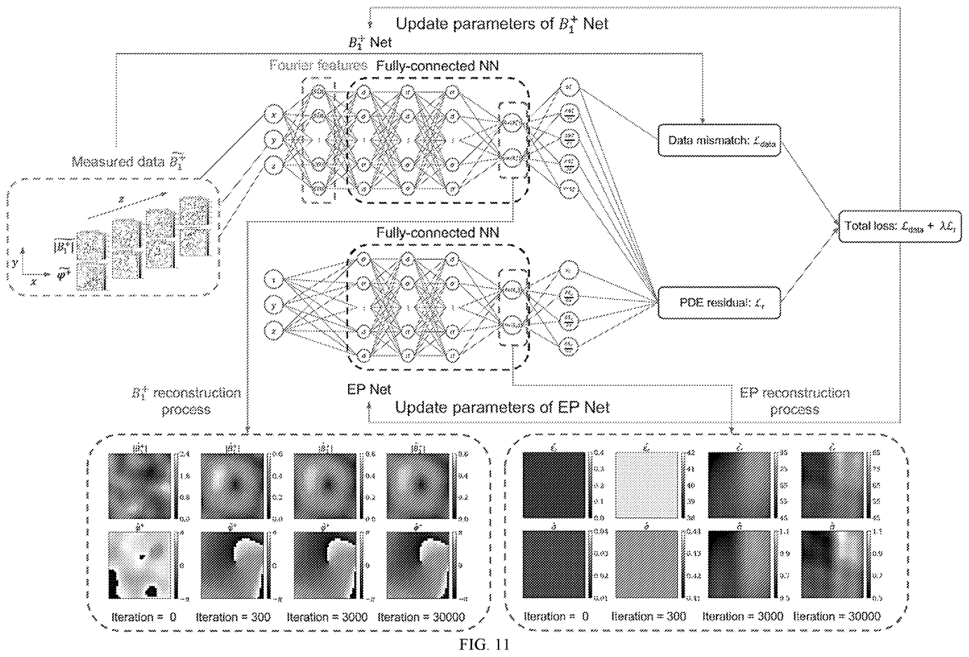

only for a limited N locations rᵢ=(xᵢ, yᵢ, zᵢ). The workflow of PIFON-EPT can be summarized in FIG. 11.

FIG. 11 illustrates PIFON-EPT workflow. Two separate fully-connected neural networks

$${B}_{1}^{+} Net ( {B}_{1}^{+} ( r ; {θ}_{1} ) )$$

and EP Net (ε_{c}(r; θ₂)) can be defined to represent the

$${B}_{1}^{+}$$

field and the EP distributions, respectively. The

$${B}_{1}^{+}$$

Net and EP Net can be trained jointly by minimizing a composite loss function that aims to fit the measured

$${\tilde{B}}_{1}^{+}$$

data while also penalizing the partial differential equation (PDE) residual. Once trained, the resulting physics-informed

$${B}_{1}^{+}$$

Net and EP Net can be used to obtain physically consistent predictions of

$${B}_{1}^{+}$$

and EP at any arbitrary 3D location. A representative axial cut of the outputs of the neural networks obtained at different iterations during training is shown at the bottom.

EPT methods based on finite difference ap-proximation of derivatives of

$${B}_{1}^{+}$$

can lead to noise amplifications in the reconstructed EP distributions. To prevent this, the systems and methods of the present disclosure can solve an optimization problem constrained by the measured data and physical laws using physics-informed deep learning. The Helmholtz equation that describes the physical laws that must be satisfied by

$${B}_{1}^{+}$$

can be denoted in the general form on a d-dimension domain Ω∈ [symbol] as Eq. 11:

$$[ {B}_{1}^{+} ; ε ( r ) ] ( r ) = 0 , \qquad Eq . 11$$

where r∈ [symbol] is a spatial coordinate and [symbol] [⋅;ε] is a symbolic representation of the Helmholtz Eq. 7 or Eq. 9. ε(r) denotes the complex-valued EP at the location r and

$${B}_{1}^{+} ( r )$$

describes the hidden

$${B}_{1}^{+}$$

field solution governed by equation (8).

Given noisy and/or incomplete measurements

$$\{ {r}_{i} , {\tilde{B}}_{1}^{+} ( {r}_{i} ) \} \frac{N}{i = 1} ,$$

the EP distributions ε and the

$${B}_{1}^{+}$$

can be learned for all r. To do so, a Fourier neural network

$${B}_{1}^{-} ( r ; {θ}_{1} ) ,$$

constructed by Gaussian random Fourier features followed by a fully-connected neural network with a set of weights and biases θ₁, can be constructed to represent the complex

$${B}_{1}^{+}$$

field. The Gaussian random Fourier features mapping can be defined as:

$$γ ( r ) = [ \begin{matrix}cos ( Br ) \\ sin ( Br )\end{matrix} ] \qquad Eq . 12$$

where each entry in B∈R^{m×3} is sampled from a Gaussian distribution [symbol] (0, σ²). 2m equals the width of the fully-connected neural network following the defined Fourier features and σ>0 is a task-specific hyperparameter. An additional fully-connected neural network ε_{c}(r; θ₂) with independent weights and biases θ₂ can be used to estimate the distribution or EP. Hereinafter,

$${B}_{1}^{+}$$

(r; θ₁) and ε_{c}(r; θ₂) can be referred to as

$${B}_{1}^{+}$$

net and EP net, respectively.

The PDE residual of Eq. 8 can be transformed to:

$$ℛ ( r , {θ}_{1} , {θ}_{2} ) := [ {ℬ}_{1}^{+} ( r , {θ}_{1} ) ; {ε}_{c} ( r , {θ}_{2} ) ] ( r ) \qquad Eq . 13$$

A good set of candidate parameters {θ₁, θ₂} can be obtained by minimizing the following composite loss function via gradient descent with the Adam optimizer:

$$ℒ ( {θ}_{1} , {θ}_{2} ) = {ℒ}_{data} ( {θ}_{1} ) + {λℒ}_{r} ( {θ}_{1} , {θ}_{2} ) , \qquad Eq . 14$$

$${ℒ}_{data} ( {θ}_{1} ) = \frac{1}{N} {\sum }_{i = 1}^{N} {\vert Re \{ {ℬ}_{1}^{+} ( {r}_{i} ; {θ}_{1} ) \} - Re \{ {\tilde{ℬ}}_{1}^{+} ( {r}_{i} ) \} \vert}^{2} + \frac{1}{N} {\sum }_{i = 1}^{N} {\vert Im \{ {ℬ}_{1}^{+} ( {r}_{i} ; {θ}_{1} ) \} - Im \{ {\tilde{ℬ}}_{1}^{+} ( {r}_{i} ) \} \vert}^{2} ,$$

$${ℒ}_{r} ( {θ}_{1} , {θ}_{2} ) = \frac{1}{N} {\sum }_{i = 1}^{N} {\vert ℛ ( {r}_{i} , {θ}_{1} , {θ}_{2} ) \vert}^{2}$$

[symbol] _{data} denotes the data mismatch and [symbol] denotes the PDE residual. λ denotes the weight coefficient in the loss function, which balances the two loss terms in the composite loss. λ is a hyperparameter that can either be specified by the user or be tuned automatically. All the derivatives of

$${B}_{1}^{+}$$

(r, θ₁) and ε_{c} (r; θ₂) with respect to the spatial coordinate r as well as the gradient of the loss function with respect to the neural network parameters {θ₁, θ₂}, can be computed using automatic differentiation algorithms.

The workflow of the PIFON-EPT (FIG. 11) can be summarized as follows. First, two separate fully-connected neural networks

$${B}_{1}^{+}$$

Net and EP Net (ε_{c}(r; θ₂)) can be defined to represent the

$${B}_{1}^{+}$$

and the EP, respectively. A random Fourier features mapping can be embedded into

$${B}_{1}^{+}$$

Net to learn high frequency components of the target

$${B}_{1}^{+}$$

field solution more efficiently. Second,

$${B}_{1}^{+}$$

Net and EP Net can be trained jointly by minimizing a composite loss function that aims to fit the measured

$${\tilde{B}}_{1}^{+}$$

data, while satisfying the physics laws characterized by the PDE residual. Finally, the trained physics-informed

$${B}_{1}^{+}$$

Net and EP Net can be used to obtain physically consistent predictions of

$${B}_{1}^{+}$$

and EP at arbitrary 3D locations.

If piece-wise constant EP is assumed, then the Helmholtz equation simplifies as in Eq. 7. Eq. 9 is a generalized form of the same equation, which accounts for gradients of EP, but is yet not fully general because to reduce the number of unknowns, B_{Z} is assumed to be equal to zero. Depending on which Helmholtz equation is used, there can be two variants of PIFON-EPT: simplified PIFON-EPT and generalized PIFON EPT.

For simplified PIFON-EPT, piece-wise constant EP is assumed and it does not require any assumption on B_{Z}. Following Eq. 7, the Helmholtz residual (Eq. 13) can be represented as:

$${ℛ}_{H} = {∇}^{2} {ℬ}_{1}^{+} ( r ; {θ}_{1} ) + {κ}_{0}^{2} {ε}_{c} ( r ; {θ}_{2} ) {ℬ}_{1}^{+} ( r ; {θ}_{1} ) \qquad Eq . 15$$

For generalized PIFON-EPT, B_{Z}≈0 and it uses the generalized Eq. 9. The Helmholtz residual (Eq. 13) becomes:

$${ℛ}_{GH} = {∇}^{2} {ℬ}_{1}^{+} ( r ; {θ}_{1} ) + {κ}_{0}^{2} {ε}_{c} ( r ; {θ}_{2} ) {ℬ}_{1}^{+} ( r ; {θ}_{1} ) - \frac{1}{{ε}_{c} ( r ; {θ}_{2} )} \qquad Eq . 16$$

$$( \frac{\partial {ℬ}_{1}^{+} ( r ; {θ}_{1} )}{\partial x} - i ( \frac{\partial {ℬ}_{1}^{+} ( r ; {θ}_{1} )}{\partial y} ) ) ( \frac{\partial {ε}_{c} ( r ; {θ}_{2} )}{\partial x} + i \frac{\partial {ε}_{c} ( r ; {θ}_{2} )}{\partial y} ) - \frac{1}{{ε}_{c} ( r ; {θ}_{2} )} ( \frac{\partial {ℬ}_{1}^{+} ( r ; {θ}_{1} )}{\partial z} ) \cdot ( \frac{\partial {ε}_{c} ( r ; {θ}_{2} )}{\partial z} ) .$$

Both techniques (e.g., simplified PIFON-EPT and generalized PIFON-EPT) can rely on knowledge of the absolute phase of

$${B}_{1}^{+} ,$$

which for a quadrature birdcage coil can be estimated from the transceive phase assumption. Note that with a sufficient number of transmit-receive coils, it is theoretically possible to solve for both the unknown absolute phase and B_{z} although the lack of suitable multi-channel coils and the computational complexity of such solution can prevent practical implementations.

A series of numerical examples can be presented to demonstrate the effectiveness of the PIFON-EPT framework. Throughout all experiments, unless otherwise specified, simulated complex

$${B}_{1}^{+}$$

maps as measured data can be used. The maps can be corrupted with white Gaussian noise with a standard deviation equal to the ratio of the pean value of

$$\vert {B}_{1}^{+} \vert$$

to a prescribed peak signal-to-noise-ratio (SNR) value. The simulations can be performed with the volume and the volume-surface integral equation methods. The volume equations can be solved using higher-order polynomials as basis functions to ensure accuracy in the

$${B}_{1}^{+}$$

distributions. All experiments can be performed on a server running Ubuntu 20.04.3 LTS operating system, with an Intel® Xeon® Silver 4216 CPU at 2.10 GHz, 64 cores, 2 threads per core, and an NVIDIA RTX 3090 GPU with 24 GB of memory.

To verify the method, a complex

$${B}_{1}^{+}$$

map obtained from the Mie Scattering theory for an infinitely long homogeneous dielectric cylinder of relative permittivity 3 and electric conductivity 0.01 S/m can be used. The operating wavelength can be λ=2.437 m and the cylinder can have a radius r equal to the wavelength. A TMz planewave can be used as the excitation.

For data acquisition, the

$${B}_{1}^{+}$$

field distribution in the domain [−2r, 2r]×[−2r, 2r] using Mie scattering theory can be computed. The pixel isotropic resolution can be set to 0.05λ. The synthetic

$${B}_{1}^{+}$$

field can be corrupted with Gaussian noise of peak SNR of 200 and then the noisy field with the peak value of

$$\vert {B}_{1}^{+} \vert$$

can be scaled to obtain synthetic

$${\tilde{B}}_{1}^{+}$$

measurements. Ine resulting

$${\tilde{B}}_{1}^{+}$$

fields can be used as the measured data for PIFON-EPT.

The PIFON training settings can include a

$${B}_{1}^{+}$$

Net constructed by a Fourier features mapping initialized with σ=2 as a coordinate embedding of the input, followed by a fully-connected neural network with 3 layers, 128 units per layer. EP Net can be constructed using a fully-connected neural network with 3 layers, 128 units per layer. The activation functions can be set as the Sine function. λ can be set to 10⁻⁴ in Eq. 14.

$${B}_{1}^{+}$$

Net and EP Net can be trained jointly using the Adam optimizer for 120 k iterations in total, with a decaying schedule of learning rates 10⁻³, 10⁻⁴, 10⁻⁵ decreased every 40 k iterations. This can take 30 minutes and 40 minutes for employing simplified PIFON-EPT and generalized PIFON-EPT, respectively.

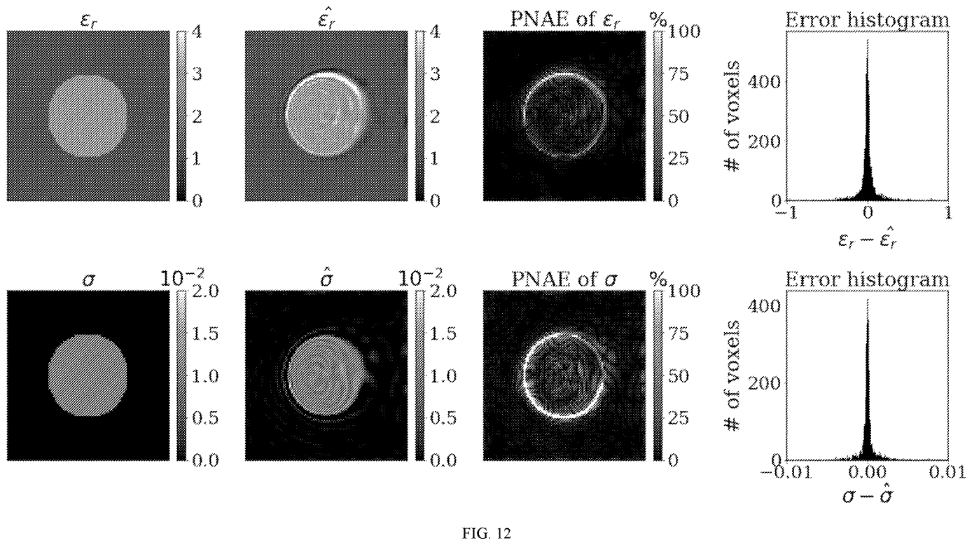

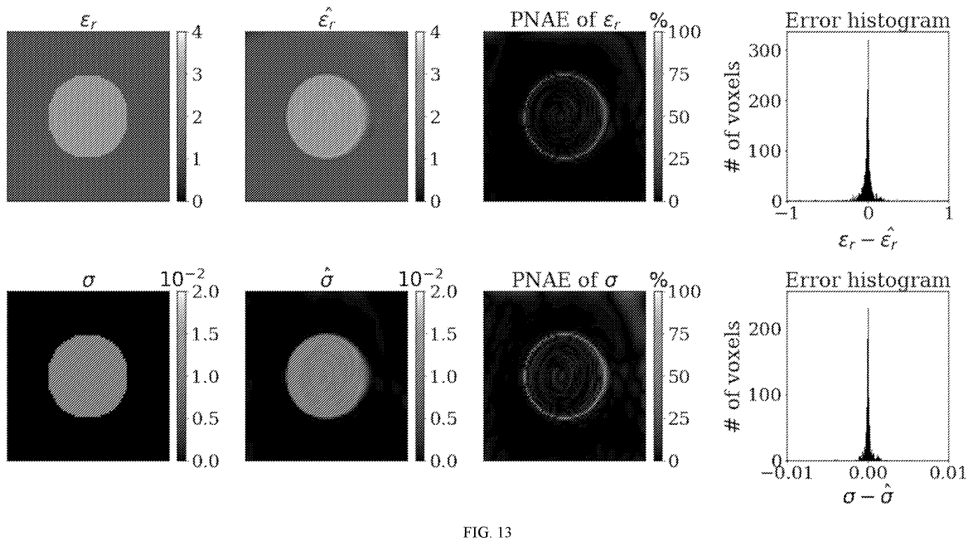

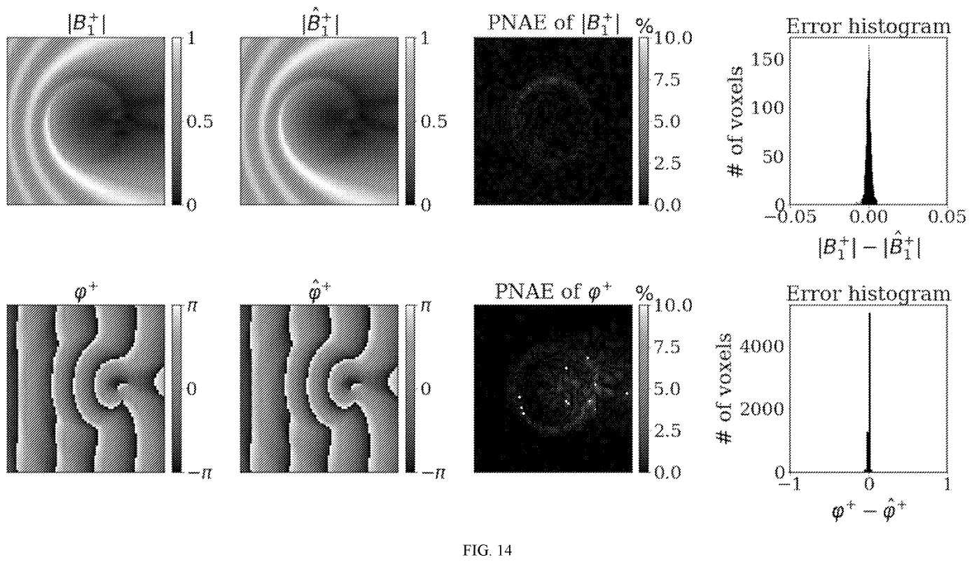

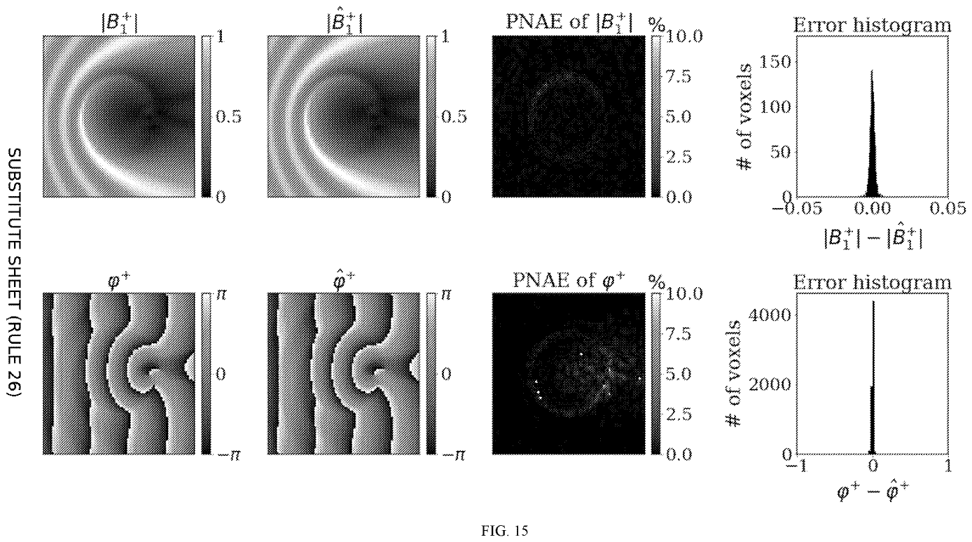

The performance of simplified PIFON-EPT and generalized PIFON-EPT can be tested using the same training settings. FIG. 12 and FIG. 13 compare the EP reconstructions against the ground truth EP for simplified PIFON-EPT and generalized PIFON-EPT, respectively. FIG. 14 and FIG. 15 compare ground truth and reconstructed

$${B}_{1}^{+}$$

for simplified PIFON-EPT and generalized PIFON-EPT, respectively. The average PNAE over the domain for the relative permittivity, conductivity, and

$${B}_{1}^{+}$$

can be 3.96%, 9.67% and 0.22%, respectively for simplified PIFON-EPT. The average PNAE over the domain for the relative permittivity, conductivity, and

$${B}_{1}^{+}$$

can be 1.80%, 1.11% and 0.20%, respectively for generalized PIFON-EPT. The generalized PIFON-EPT can achieve smaller errors near the boundary for EP reconstructions.

FIG. 12 illustrates EP reconstruction with simplified PIFON-EPT for a uniform dielectric cylinder. FIG. 12 illustrates, from left to right, ground truth EP, including relative permittivity (top) and conductivity (bottom), predicted EP using

$${\tilde{B}}_{1}^{+}$$

measurements with peak SNR of 200, peak-normalized absolute errors, distribution of the error in 6561 voxels.

FIG. 13 illustrates EP reconstruction with generalized PIFON-EPT for a uniform dielectric cylinder. FIG. 13 illustrates, from left to right, ground truth EP, including relative permittivity (top) and conductivity (bottom), predicted EP using

$${\tilde{B}}_{1}^{+}$$

measurements will peak SNR of 200, peak-normalized absolute errors, distribution of the error in 6561 voxels.

FIG. 14 illustrates reconstructed

$${B}_{1}^{+}$$

with simplified PIFON-EPT inside a uniform dielectric cylinder. FIG. 14 illustrates, from left to right, ground truth noise-free synthetic

$${B}_{1}^{+} ,$$

including magnitude (top) and transmit phase (bottom), reconstructed

$${B}_{1}^{+}$$

from noisy synthetic

$${\tilde{B}}_{1}^{+}$$

measurements with peak SNR 200, peak-normalized absolute errors, distribution of the error in 6561 voxels.

FIG. 15 illustrates reconstructed

$${B}_{1}^{+}$$

with generalized PIFON-EPI for a uniform dielectric cylinder. FIG. 15 illustrates, from left to right, ground truth noise-nice synthetic

$${B}_{1}^{+} ,$$

including magnitude (top) and transmit phase (bottom), reconstructed

$${B}_{1}^{+}$$

from noisy synthetic

$${\tilde{B}}_{1}^{+}$$

measurements with peak SNR 200, peak-normalized absolute errors, distribution of the error in 6561 voxels.

For data acquisition, the volume-surface integral equation method can be used to simulate the circularly polarized (CP) mode of the birdcage coil loaded with the cylindrical phantom at 3 T. The resolution can be set to 2 mm³.

$${B}_{1}^{+} and {B}_{1}^{-}$$

from tie central region of the cylinder (12×12×2 cm³, MR measurements out of cylindrical phantom were not used) can be used and they can be corrupted with Gaussian noise of peak SNR of 200. THE complex

$${B}_{1}^{+}$$

can be approximated using the transceive phase assumption (TPA) and the MR measurements

$$\vert {\tilde{B}}_{1}^{+} \vert$$

and φ^{˜±} can be constructed.

For the PIFON training settings, the B_{z} field of a birdcage can be negligible around the mid-plane of the coil. For this reason, the generalized PIFON-EPT can be used to perform the reconstruction. For

$${B}_{1}^{+}$$

Net, the Fourier feature mapping can be initialized with σ=40 as a coordinate embedding of the input, followed by a fully-connected neural network with 6 layers, 128 units per layer. EP Net can be an additional Fourier neural network constructed by a Fourier feature mapping initialized with σ=2, followed by a fully-connected neural network with 6 layers, 128 units per layer. All the activation functions can be set as the Sine function and set λ=10-8 in equation (11).

$${B}_{1}^{+}$$

Net and EP Net can be trained jointly using the Adam optimizer for 120 k iterations in total, with a decaying schedule of learning rates 10⁻³, 10⁻⁴, 10⁻⁵ decreased every 40 k iterations. The overall training time can take 220 minutes on the GPU.

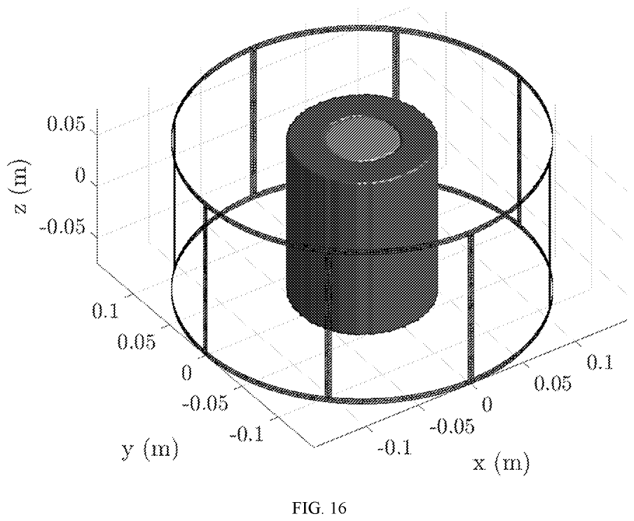

A two-compartment concentric cylindrical phantom with relative permittivity ε={70, 78} and conductivity σ={0.5, 1} S/m (outer, inner) can be considered. The cylinder can load a high-pass birdcage coil with eight legs as shown in FIG. 16. FIG. 16 illustrates the geometry of the high-pass birdcage coil loaded with a two-compartments cylindrical phantom. The outer and inner radius of the cylinder can be 6 cm and 3 cm, respectively, and its length can be 14 cm. For this example, the PIFON-EPT can be compared with the Helmholtz-EPT (H-EPT) and the convection-reaction EPT (CR-EPT). In particular, the implementations in EPTlib, with the Savitzky-Golay filter with an ellipsoid-shaped kernel of size 2×2×2 can be used to approximate all the gradients. For CR-EPT, the diffusion coefficient can be set to 0.02 and the conductivity boundary condition can be set to 0.55 S/m.

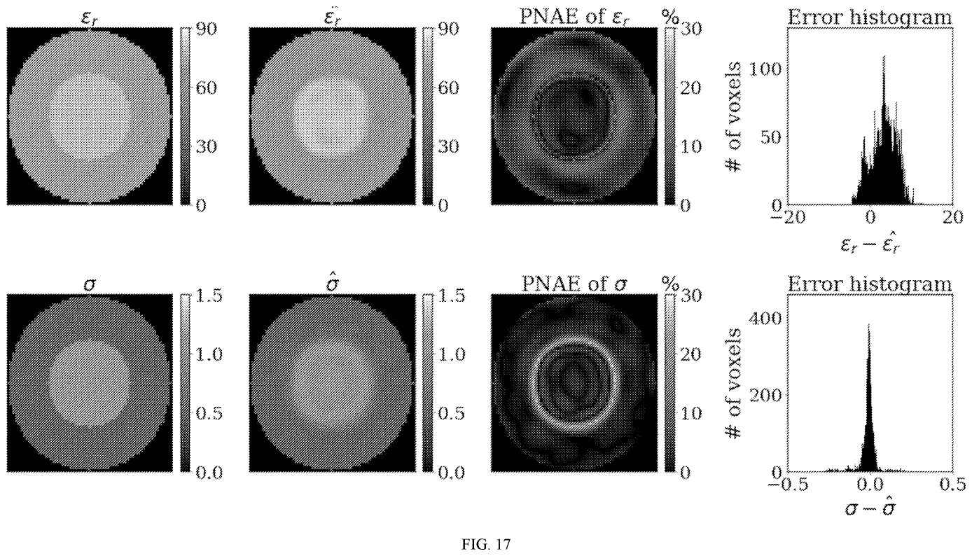

Results are shown in FIG. 17 for the central axial cut of the cylinder. The average PNAE over the entire volume of the cylinder can be 4.84%, 3.20% and 0.25% for relative permittivity, conductivity and

$${B}_{1}^{+} ,$$

respectively. FIG. 17 illustrates EP reconstructed with generalized PIFON-EPT for the two-compartment cylindrical phantom. FIG. 17 illustrates, from left to right, ground truth EP for the central axial cut of the phantom, including relative permittivity (top) and conductivity (bottom), estimated EP using synthetic measurements

$${\tilde{B}}_{1}^{+}$$

measurements with peak SNR of 200, peak-normalized absolute errors, distribution of the error in 31031 voxels.

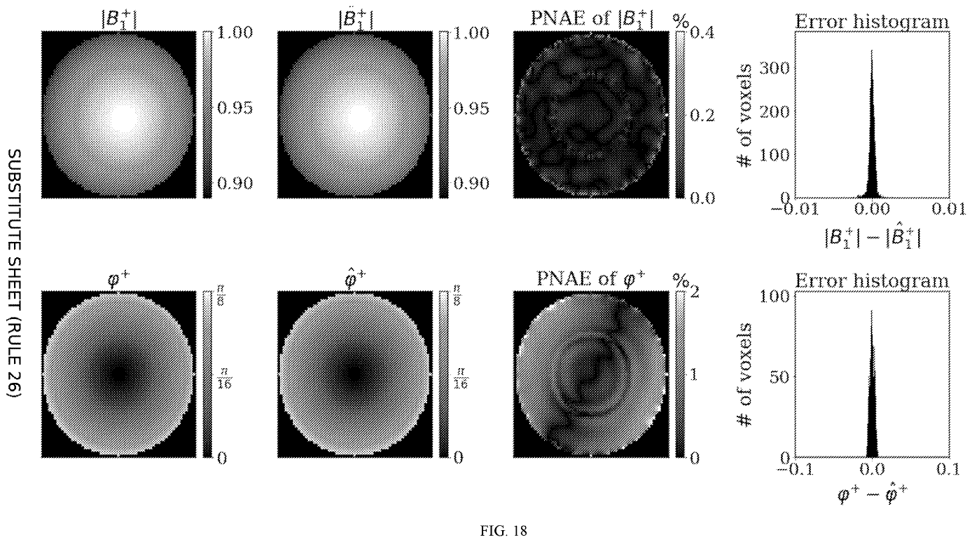

FIG. 18 illustrates reconstructed

$${B}_{1}^{+}$$

with generalized PIFON-EPT for the two-compartment cylindrical phantom. FIG. 18 illustrates, from left to right, noise-tree synthetic

$${B}_{1}^{+}$$

for the central axial cut, including magnitude (top) and transmit phase (bottom), reconstructed

$${B}_{1}^{+}$$

field from noisy

$${\tilde{B}}_{1}^{+}$$

measurements, peak-normalized absolute errors, distribution of the error in 31031 voxels.

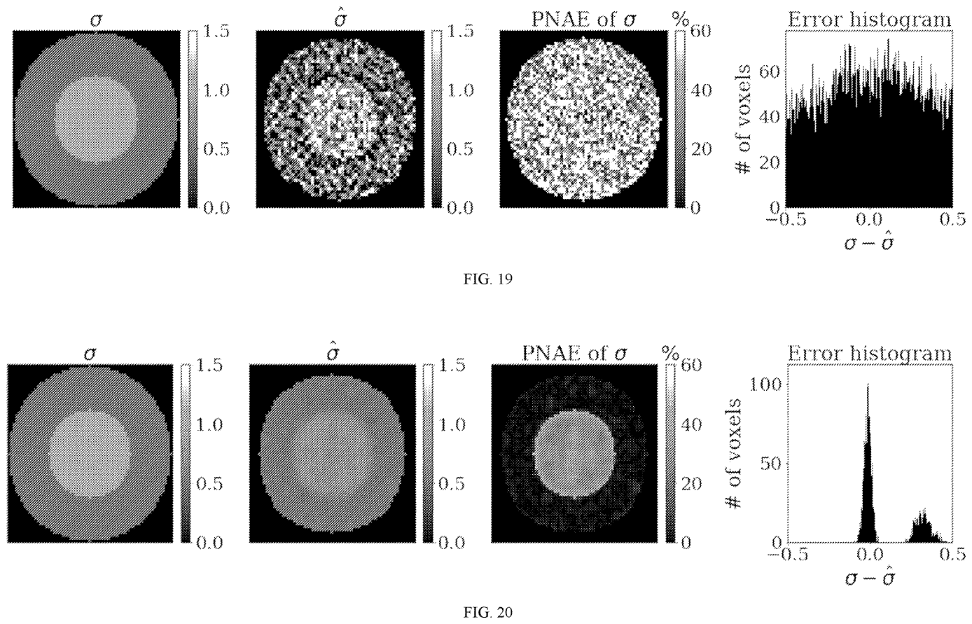

FIG. 19 and FIG. 20 show the conductivity reconstruction results for H-EPT and CR-EPT, respectively, along with the PNAE distribution and the error histogram. The average PNAE over the volume where EP was reconstructed can be 51.80% and 11.28% for H-EPT and CR-EPT, respectively.

FIG. 19 illustrates conductivity reconstructed with phase-based H-EPT for the two-compartment cylindrical phantom. FIG. 19 illustrates, from left to right, ground truth conductivity for the central axial cut of the phantom, estimated conductivity using φ^{˜±} measurements with peak SNR of 200, the peak-normalized absolute errors, the distribution of the error in 17423 voxels.

FIG. 20 illustrates conductivity reconstructed with phase-based CR-EPT for the two-compartment cylindrical phantom. From left to right, ground truth conductivity for the central axial cut of the phantom, estimated conductivity using q^{˜±} measurements with peak SNR of 200, the peak-normalized absolute errors, the distribution of the error in 11645 voxels.

In the example of a four-compartment phantom, the performance of PIFON-EPT at 7 tesla can be explored. A tissue-mimicking four-compartment phantom shaped as a 20×20×20 cm³ rectangular parallelepiped can be considered. The relative permittivity values of the four compartments can be 51, 56, 65, and 76. The corresponding electric conductivity values can be 0.56, 0.69, 0.84, and 1.02 S/m, respectively.

For data acquisition, a single external excitation can be used to illuminate the phantom, generated from a numerical electro-magnetic basis. 6 mm isotropic voxel resolution can be used. The synthetic

$${B}_{1}^{+}$$

can be corrupted with different levels of Gaussian noise (Peak SNR=200, 100, 50, 20) and then each field map can be scaled by the corresponding peak value of

$$\vert {B}_{1}^{+} \vert$$

to obtain synthetic

$${\tilde{B}}_{1}^{+}$$

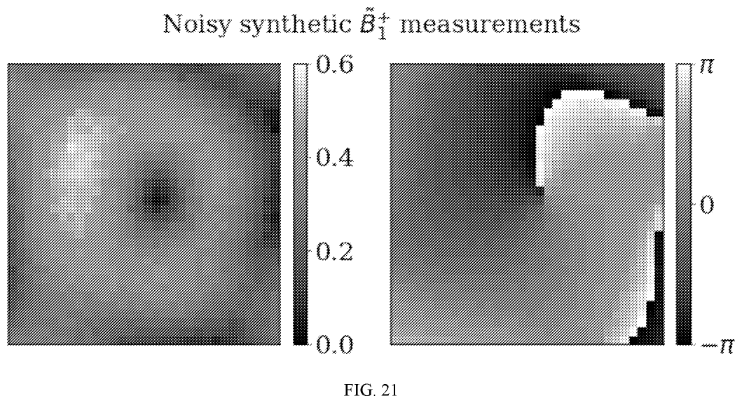

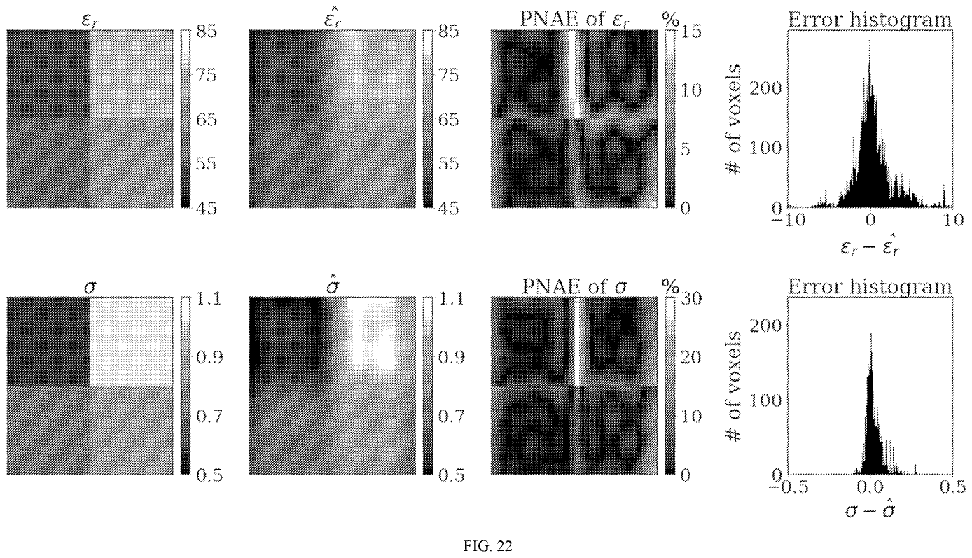

measurements. The case of peak SNR=50 is shown in FIG. 21.

FIG. 21 illustrates noisy synthetic

$${\tilde{B}}_{1}^{+}$$

measurements. Magnitude (left) and transmit phase (right) are shown for the central axial cut of the four-compartment phantom. The peak SNR can be set to 50.

For the PIFON training settings, since the B₁ field in the z₁ direction cannot be assumed zero at 7 T, the simplified PIFON-EPT can be used. The

$${B}_{1}^{+}$$

Net can be constructed using a Fourier feature mapping initialized with σ=40 as a coordinate embedding of the input, followed by a fully-connected neural network with 3 layers, 128 units per layer. For EP Net, a second fully-connected neural network with 3 layers, 128 units per layer, can be used. All the activation functions can be set as the Sine function. λ can be 10⁻⁸ in Eq. 14.

$${B}_{1}^{+}$$

Net anu EP Net can be trained jointly using the Adam optimizer for 30 k iterations in total, with a decaying schedule of learning rates 10⁻³, 10⁻⁴, 10⁻⁵ decreased every 10 k iterations, which can take 21.4 minutes on the GPU.

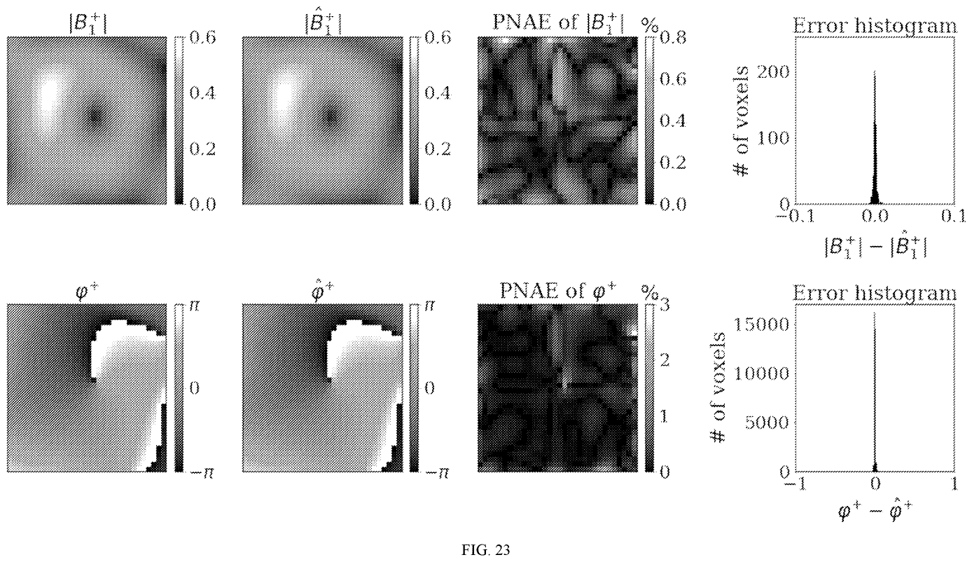

FIGS. 22 and 23 show the results for the central slice of the four-compartment phantom. The method can remove the noise from the noisy synthetic measurements (FIG. 21) and the reconstructed

$${B}_{1}^{+}$$

can be qualitatively similar to be noise-free ground truth. The average PNAE over the volume can be 2.47%, 4.01%, 0.24% for the relative permittivity, conductivity and

$${B}_{1}^{+} ,$$

respectively.

FIG. 22 illustrates EP reconstructed with simplified PIFON-EPT for the four-compartment phantom. FIG. 22 illustrates, from left to right, ground truth EP for the central axial cut of the phantom, including relative permittivity (top) and conductivity (bottom), EP reconstructed from synthetic

$${\tilde{B}}_{1}^{+}$$

measurements with peak SNR 50, peak-normalized absolute errors, error distribution in 32768 voxels.

FIG. 23 illustrates reconstructed

$${B}_{1}^{+}$$

with simplified PIFON-EPI for the four-compartment phantom. FIG. 23 illustrates, from left to right, ground truth synthetic

$${B}_{1}^{+}$$

for the central axial cut of the phantom, including magnitude (top) and transmit phase (bottom), reconstructed

$${B}_{1}^{+}$$

field from noise-corrupted synthetic

$${\tilde{B}}_{1}^{+}$$

measurements with peak SNR of 50, the peak-normalized absolute errors, the distribution of the error in 32768 voxels.

The average PNAE for the reconstructed EP and

$${B}_{1}^{+}$$

for different levels of noise in the synthetic measurements are summarized in Table 1. The reconstructions can be robust for a wide range of noise levels.

TABLE 1
Robustness analysis of PIFON-EPT with respect to the noise level.

| | Peak SNR | | | |
|---|---|---|---|---|
| PNAE | 200 | 100 | 50 | 20 |
| εᵣ | 2.56% | 2.64% | 2.47% | 2.56% |
| σ | 4.00% | 4.10% | 4.01% | 3.96% |
| ${B}_{1}^{+}$ | 0.15% | 0.17% | 0.24% | 0.49% |

In the numerical experiment of an incomplete four-compartment phantom, the same four-compartment phantom as before can be used, but the synthetic

$${\tilde{B}}_{1}^{+}$$

measurements can be assumed to be incomplete, which could happen in reality if the measured signal is too low or corrupted for certain voxels to reconstruct

$${B}_{1}^{+} .$$

Whether PIFON-EPT could reconstruct the EP and a complete, denoised

$${B}_{1}^{+}$$

for the entire volume can be tested.

For data acquisition, from 20% to 90% of the voxels in the synthetic

$${\tilde{B}}_{1}^{+}$$

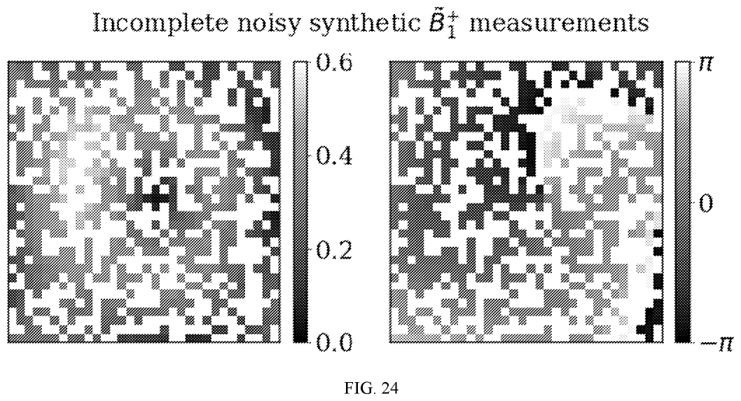

measurements with peak SNR of 50 can be randomly set to zero. As a result, only 10% to 80% of the measurements were used as input for simplified PIFON-EPT. FIG. 24 shows one of the resulting

$${\tilde{B}}_{1}^{+}$$

measurements for the central axial cut, where 50% of the

$${\tilde{B}}_{1}^{+}$$

values were set to zero. FIG. 24 illustrates incomplete noisy synthetic

$${\tilde{B}}_{1}^{+}$$

measurements with 50% of the voxels set to zero. Magnitude (left) and transmit phase (right) are shown for the central axial cut of the four-compartment phantom. The peak SNR can be set to 50.

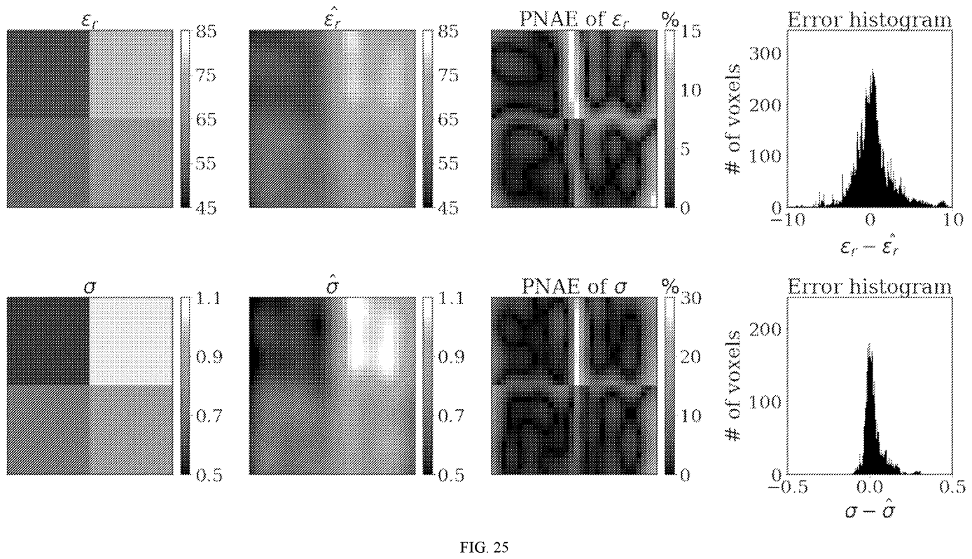

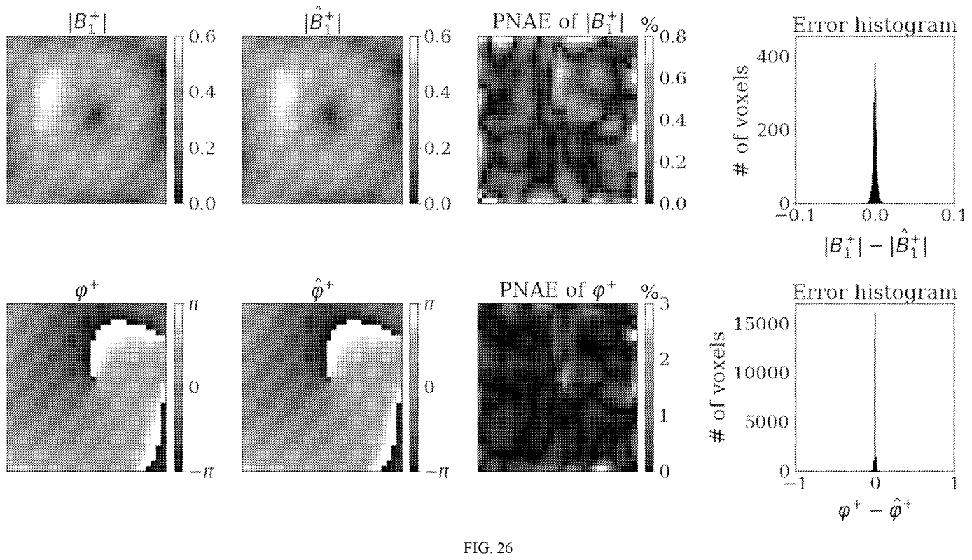

The same training settings as for the previous experiment can be used. The total training time when 10%, 20%, 50%, and 80% of the measurements were used can be 10, 11, 15, and 18 minutes, respectively. FIGS. 25 and 26 show the ground truth EP and noise-free synthetic

$${B}_{1}^{+}$$

(first column), the reconstructed EP and the denoised completed

$${B}_{1}^{+}$$

(second column), the PNAE of the predicted EP and

$${B}_{1}^{+}$$

(third column) and their error distribution over the entire volume of the phantom (fourth column), respectively. The figures show the case where only 50% of the synthetic

$${\tilde{B}}_{1}^{+}$$

measurements were use. The method can accurately reconstruct the EP and

$${B}_{1}^{+}$$

for the whole domain, despite using partial measurement as the input. The average PNAE over the entire volume of the phantom can be 2.49%, 4.09% and 0.32% for the relative permittivity, conductivity, and

$${B}_{1}^{+} ,$$

respectively.

FIG. 25 illustrates reconstructed EP with simplified PIFON-EPT for the incomplete four-compartment phantom. FIG. 25 illustrates, from left to right, ground truth EP for the central axial cut of the phantom, including relative permittivity (top) and conductivity (bottom), estimated EP using 50% of

$${\tilde{B}}_{1}^{+}$$

with peak SNR of 50, the peak-normalized absolute errors, the distribution of the error in 32768 voxels.

FIG. 26 illustrates reconstructed

$${B}_{1}^{+}$$

with simplified PIFON-EPT for the incomplete four-compartment phantom. FIG. 26 illustrates, from left to right, magnitude (top) and transmit phase (bottom) of the synthetic

$${B}_{1}^{+}$$

field for the central axial cut of the phantom, reconstructed

$${B}_{1}^{+}$$

field using 50% of

$${\tilde{B}}_{1}^{+}$$

with peak SNR of 50, peak-normalized absolute errors, error distribution in 32768 voxels.

Table 2 summarizes the average PNAE for the EP and

$${B}_{1}^{+}$$

when different percentages of the synthetic measurements are used. The error for the

$${B}_{1}^{+}$$

reconstruction can increase when a smaller percentage of the data was used. However, PIFON-EPT can yield robust results until as little as 20% of the measurements was used as input.

TABLE 2
Performance of PIFON-EPT with respect to the percentage of
measurements used as input for the reconstructions.

| | % of the Data | | | |
|---|---|---|---|---|
| PNAE | 80% | 50% | 20% | 10% |
| εᵣ | 2.41% | 2.49% | 2.77% | 7.22% |
| σ | 3.94% | 4.09% | 4.06% | 7.58% |
| ${B}_{1}^{+}$ | 0.26% | 0.32% | 0.57% | 2.69% |

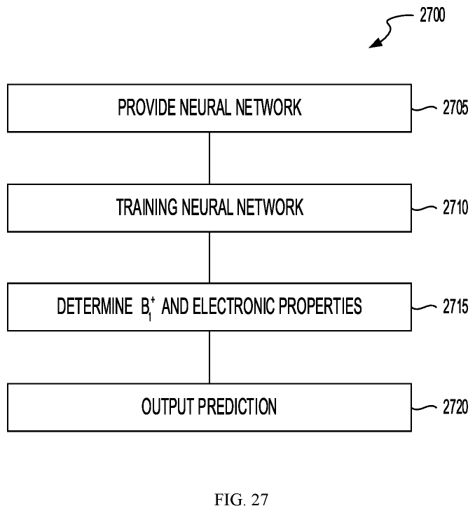

FIG. 27 illustrates a method 2700. The method 2700 can be for electrical property tomography. The method 2700 can include providing one or more neural networks (BLOCK 2705). The method 2700 can include training the one or more neural networks (BLOCK 2710). The method 2700 can include determining a prediction of

$${B}_{1}^{+}$$

and EP (BLOCK 2715). Ine method 2700 can include outputting the prediction (BLOCK 2720).

The method 2700 can include providing one or more neural networks (BLOCK 2705). The one or more neural networks can include a first neural network. The first neural network can represent

$${B}_{1}^{+}$$

(e.g., magnetic field). For example, the first neural network can represent magnetic field.

The one or more neural networks can include a second neural network. The second neural network can represent electrical properties (EP). For example, the second neural network can represent permittivity or electric conductivity. Electrical properties can include at least one of permittivity or electric conductivity. The second neural network can be connected to the first neural network. The first neural network can be connected to the second neural network. The first neural network and the second neural network can be fully connected.

The method 2700 can include training the one or more neural networks (BLOCK 2710). For example, the method 2700 can include training the first neural network and the second neural network jointly. Training the first neural network and the second neural network jointly can include minimizing a composite loss function. The composite loss function can be configured to fit measured

$${\tilde{B}}_{1}^{+}$$

data. The composite loss function can include:

$$ℒ ( {θ}_{1} , {θ}_{2} ) = {ℒ}_{data} ( {θ}_{1} ) + {λℒ}_{r} ( {θ}_{1} , {θ}_{2} ) ,$$

$${ℒ}_{data} ( {θ}_{1} ) = \frac{1}{N} {\sum }_{i = 1}^{N} {\vert Re \{ {ℬ}_{1}^{+} ( {r}_{i} ; {θ}_{1} ) \} - Re \{ {\tilde{ℬ}}_{1}^{+} ( {r}_{i} ) \} \vert}^{2} + \frac{1}{N} {\sum }_{i = 1}^{N} {\vert Im \{ {ℬ}_{1}^{+} ( {r}_{i} ; {θ}_{1} ) \} - Im \{ {\tilde{ℬ}}_{1}^{+} ( {r}_{i} ) \} \vert}^{2} , {ℒ}_{r} ( {θ}_{1} , {θ}_{2} ) = \frac{1}{N} {\sum }_{i = 1}^{N} {\vert ℛ ( {r}_{i} , {θ}_{1} , {θ}_{2} ) \vert}^{2} .$$

The method 2700 can include computing θ₁ and θ₂ using one or more automatic differentiation algorithms.

The method 2700 can include determining a prediction of

$${B}_{1}^{+}$$

EP (BLOCK 2715). For example, the method 2700 can include determining a prediction

$${B}_{1}^{+}$$

and EP at one or more predetermined locations. The method 2700 can include determining, from the trained first neural network and the trained second neural network, a prediction of

$${B}_{1}^{+}$$

and EP at the one or more predetermined locations.

The method 2700 can include outputting the prediction (BLOCK 2720). For example, the method 2700 can include outputting the prediction of

$${B}_{1}^{+}$$

### and EP.

The method 2700 can include embedding a random Fourier features mapping into the first neural network. The method 2700 can include embedding a random Fourier features mapping into the second neural network.

The method 2700 can include estimating EP field distribution from magnetic resonance (MR) measurements. The MR measurements can be noisy (e.g., low signal to noise ratio). The MR measurements can be incomplete.

The method 2700 can include estimating magnetic transmit field distribution from magnetic resonance measurements. The MR measurements can be noisy (e.g., low signal to noise ratio). The MR measurements can be incomplete.

The method 2700 can include obtaining magnetic resonance data. The method 2700 can include inputting the MR data into the first neural network. The method 2700 can include inputting the MR data into the second neural network.

A non-transitory computer-readable media having computer-readable instructions stored thereon that when executed by a processor cause the processor to provide a first neural network connected to a second neural network, the first neural network representing

$${B}_{1}^{+}$$

and the second neural network representing electrical properties (EP), train the first neural network and the second neural network jointly, determine, from the trained first neural network and the trained second neural network, a prediction of

$${B}_{1}^{+}$$

and EP at one or more predetermined locations, and output the prediction of

$${B}_{1}^{+}$$

### and EP.

The computer-readable instructions can cause the processor to obtain magnetic resonance data. The computer-readable instructions can cause the processor to embed a random Fourier features mapping into the first neural network. The computer-readable instructions can cause the processor to minimize a loss function. The computer-readable instructions can cause the processor to compute θ₁ and θ₂ using one or more automatic differentiation algorithms. The computer-readable instructions can cause the processor to estimate one or more of EP field distribution or magnetic transmit field distribution from magnetic resonance measurements.

The systems and methods of present disclosure can reformulate EPT as a physics-constrained optimization problem with the goal to train two independent neural networks

$$( {B}_{1}^{+} Net and EP Net )$$

to represent the

$${B}_{1}^{+}$$

and EP at any location interest. To achieve that, a composite loss that aims to fit

$${\tilde{B}}_{1}^{+}$$

measurements can be minimized while penalizing the PDE residual via gradient descent with the Adam optimizer. Penalizing the PDE residual can not only help EP Net predict the EP distributions that best describe the measured data but also prevents

$${B}_{1}^{+}$$

Net from fitting the noise. Compared with other EPT methods that rely on finite differences to approximate gradients of noisy

$${\tilde{B}}_{1}^{+}$$

measurements, which is prone to noise amplifications and artifacts, PIFON-EPT can use automatic differentiation to calculate all the necessary gradients from denoised

$${B}_{1}^{+}$$

maps provided by

$${B}_{1}^{+}$$

Net. This way of computing derivaties can make the method robust to noise. Unlike other supervised deep learning-based EPT methods, the present method does not require a large amount of known data pairs to supervise the training. Compared with other hybrid deep learning EPT methods, which can combine deep learning and CR-EPT to solve EP from convection-reaction equations, our method directly trains a neural network (EP Net) to represent the EP based on measured data and the Helmholtz PDE without requiring any boundary conditions and hyperparameter tuning for the diffusion coefficient.

A concern for

$${B}_{1}^{+}$$

maps represented by neural net-works is that deep fully-connected networks could fail to learn high-frequency components of the target functions because of the spectral bias. To overcome the spectral bias and ensure that

$${B}_{1}^{+}$$

Net would efficiently learn the high-frequency details of

$${B}_{1}^{+} ,$$

Fourier features mapping can be applied as an input embedding to the

$${B}_{1}^{+} .$$

In the concentric cylindrical phantom example, Fourier features mapping can be applied to EP Net because it could help the network avoid predicting homogeneous EP distributions.

In simplified PIFON-EPT, a homogeneous distribution of EP can be assumed. This assumption can introduce errors near the interface between regions of different EP values and can deteriorate the quality of the reconstructions. When B_{Z} is negligible, the generalized PIFON-EPT can be used, which allows the estimation of inhomogeneous EP distributions based on the generalized Helmholtz equation (Eq. 9) which can greatly decrease the errors near the tissue boundaries. PIFON-EPT can return 48.6% and 8.08% more accurate results on average compared to H-EPT and CR-EPT. Furthermore, CR-EPT can require tuning of the boundary condition value and the diffusion coefficient parameter until the reconstructed conductivity is close to the ground-truth value, which may not be practical in experiments where the ground-truth values are unknown.

PIFON-EPT can be the only EPT method that can reconstruct EP and

$${B}_{1}^{+}$$

for an entire object, using incomplete and noisy

$${B}_{1}^{+}$$

measurements. This can be demonstrated for an ultra-high field MRI example, using complex-valued synthetic

$${B}_{1}^{+}$$

measurements. The same approach can be impractical in actual experiments because the absolute phase of the

$${B}_{1}^{+}$$

is not measurable and the TPA does not hold at 7 tesla. However, note that PIFON-EPT could be adapted to work with multiple transmit coils, which could provide enough degrees of freedom to enable EP reconstruction using the relative phase of

$${B}_{1}^{+}$$

between the coil channels, which can be measured.

PIFON-EPT can have a limitation when B_{z} can not be assumed equal to zero. In this case, boundary artifacts appearing in the reconstructed EP might not be eliminated. This limitation could be overcome by using multiple transmit-receive coils. B_{z} can be assumed negligible if the main field strength is lower or equal to 3T. The network's expressive power may not be enough to reconstruct both the EP and the

$${B}_{1}^{+}$$

in such a case. To address this, the network deeper can be made deeper and more complex architectures (e.g., Fourier mapping also in the EP Net) to accurately represent the EP and

$${B}_{1}^{+}$$

can be used, which can increase the network's training time. This problem could be solved by designing compressed network architectures to replace the fully-connected neural networks.

PIFON-EPT can be a technique to estimate EP and magnetic transmit field distributions from noisy and/or incomplete MR measurements. PIFON-EPT can be accurate and robust even when its input is corrupted with a significant amount of noise. Since PIFON-EPT can efficiently de-noise MR measurements, it has the potential to improve other MR-based EPT methods that rely on magnetic transmit fields as inputs. The algorithms can be used on realistic human head models and experimental validation can be performed.

Embodiments of the subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors, and any one or more processors of a digital computer. A processor can receive instructions and data from a read only memory or a random-access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information, or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the solution discussed above. The computer readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects of embodiments as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. A method, comprising:
providing a first neural network connected to a second neural network, the first neural network representing
$${B}_{1}^{+}$$
and the second neural network representing electrical properties (EP);
training the first neural network and the second neural network jointly;
determining, from the trained first neural network and the trained second neural network, a prediction of
$${B}_{1}^{+}$$
and EP at one or more predetermined locations; and
outputting the prediction of
$${B}_{1}^{+}$$
and EP.

2. The method of claim 1, further comprising embedding a random Fourier features mapping into the first neural network.

3. The method of claim 1, wherein training the first neural network and the second neural network jointly comprises minimizing a composite loss function.

4. The method of claim 3, wherein the composite loss function is configured to fit measured
$${\tilde{B}}_{1}^{+}$$
data.

5. The method of claim 3, wherein the composite loss function comprises:
$$ℒ ( {θ}_{1} , {θ}_{2} ) = {ℒ}_{data} ( {θ}_{1} ) + {λℒ}_{r} ( {θ}_{1} , {θ}_{2} ) ,$$
$${ℒ}_{data} ( {θ}_{1} ) = \frac{1}{N} {\sum }_{i = 1}^{N} {\vert Re \{ {ℬ}_{1}^{+} ( {r}_{i} ; {θ}_{1} ) \} - Re \{ {\tilde{ℬ}}_{1}^{+} ( {r}_{i} ) \} \vert}^{2} + \frac{1}{N} {\sum }_{i = 1}^{N} {\vert Im \{ {ℬ}_{1}^{+} ( {r}_{i} ; {θ}_{1} ) \} - Im \{ {\tilde{ℬ}}_{1}^{+} ( {r}_{i} ) \} \vert}^{2} ,$$
$${ℒ}_{r} ( {θ}_{1} , {θ}_{2} ) = \frac{1}{N} {\sum }_{i = 1}^{N} {\vert ℛ ( {r}_{i} , {θ}_{1} , {θ}_{2} ) \vert}^{2} .$$

6. The method of claim 3, further comprising computing θ₁ and θ₂ using one or more automatic differentiation algorithms.

7. The method of claim 1, further comprising estimating EP field distribution from magnetic resonance measurements.

8. The method of claim 1, further comprising estimating magnetic transmit field distribution from magnetic resonance measurements.

9. The method of claim 1, wherein electrical properties can include at least one of permittivity or electric conductivity.

10. The method of claim 1, wherein the first neural network and the second neural network are fully connected.

11. The method of claim 1, further comprising:
obtaining magnetic resonance (MR) data; and
inputting the MR data into the first neural network and the second neural network.

12. A non-transitory computer-readable media having computer-readable instructions stored thereon that when executed by a processor cause the processor to:
provide a first neural network connected to a second neural network, the first neural network representing
$${B}_{1}^{+}$$
presenting electrical properties (EP);
train the first neural network and the second neural network jointly;
determine, from the trained first neural network and the trained second neural network, a prediction of
$${B}_{1}^{+}$$
and EP at one or more predetermined locations; and
output the prediction of
$${B}_{1}^{+}$$
and EP.

13. The non-transitory computer-readable media of claim 12, wherein the computer-readable instructions cause the processor to obtain magnetic resonance (MR) data.

14. The non-transitory computer-readable media of claim 12, wherein the computer-readable instructions cause the processor to embed a random Fourier features mapping into the first neural network.

15. The non-transitory computer-readable media of claim 12, wherein the computer-readable instructions cause the processor to minimize a loss function.

16. The non-transitory computer-readable media of claim 12, wherein the computer-readable instructions cause the processor to compute θ₁ and θ₂ using one or more automatic differentiation algorithms.

17. The non-transitory computer-readable media of claim 12, wherein the computer-readable instructions cause the processor to estimate one or more of EP field distribution or magnetic transmit field distribution from magnetic resonance measurements.

18. A method for identifying a medical indication, comprising obtaining magnetic resonance (MR) data for an individual;
constructing an electrical property (EP) map for the individual using a machine learning model; and
identifying a medical condition in the individual based on a correlation between the EP map and medical indications.

19. The method of claim 18, wherein the machine learning model comprises a first neural network connected to a second neural network, the first neural network representing
$${B}_{1}^{+}$$
ana the second neural network representing EP.

20. The method of claim 18, further comprising treating the individual based on the identified medical condition.
